(19) United States
(12) Patent Application Publication
(10) Pub. No.: US 2025/0362306 A1
(43) Pub. Date: Nov. 27, 2025

(54) METHODS FOR ANALYZING PROTEOMIC ATTRIBUTES OF BIOLOGICAL SAMPLES, AND RELATED SYSTEMS AND APPARATUS

(71) Applicant: Calico Life Sciences LLC, South San Francisco, CA (US)

(72) Inventors: Jonathon O'Brien, San Francisco, CA (US); Anil Raj, San Francisco, CA (US); Aleksandr Gaun, San Francisco, CA (US); Adam Waite, San Francisco, CA (US); Wenzhou Li, San Francisco, CA (US); Fiona McAllister, San Francisco, CA (US)

(21) Appl. No.: 18/872,710

(22) PCT Filed: Jun. 8, 2023
(86) PCT No.: PCT/US2023/024875

§ 371 Date: Dec. 6, 2024

Related U.S. Application Data
(60) Provisional application No. 63/350,411, filed on Jun. 8, 2022.

(51) Int. Cl. G01N 33/68; G16B 25/10

(52) U.S. Cl. CPC G01N 33/6851; G01N 33/6827; G16B 25/10

(57) ABSTRACT

Disclosed herein, in some aspects, are systems and methods for processing multiplexed mass spectrometry proteomics data from a plurality of batches, each batch comprising a plurality of samples that each comprise one or more peptides. The systems and methods include receiving proteomics data and corresponding covariate values for one or more covariates. In some embodiments, for each parameter of a statistical model, a computation is performed to estimate said respective parameter, wherein each parameter represents an association between the proteomics data and the covariates. In some embodiments, each computation comprises incorporating bridge sample data to account for scan to scan variation between batches. In some embodiments, the statistical model is fitted to weighted proteomics data, thereby outputting an estimate of the parameter and one or more p-values of one or more hypothesis tests for the parameter.

### CROSS-REFERENCE TO RELATED APPLICATIONS

The present application claims the benefit of and priority to U.S. Patent Application No. 63/350,411, filed Jun. 8, 2022, which is incorporated by reference herein in its entirety.

### FIELD OF TECHNOLOGY

The present disclosure relates generally to techniques for analyzing proteomic attributes of biological samples and, more specifically, to techniques for analyzing biological samples based on the relative abundance of peptides in the samples.

### BACKGROUND

The mass spectrometer is a tool that measures the properties (e.g., mass) of a sample (e.g., molecule) by imparting an electrical charge to the sample, converting the resulting flux of electrically charged ions into a proportional electrical signal, and detecting that signal. Mass spectrometry has both qualitative and quantitative uses, including identifying unknown compounds; determining the isotopic composition of elements in a molecule; determining the structure of a compound by observing its fragmentation; quantifying the amount of a compound in a sample; determining physical, chemical, and/or biological properties of compounds; and characterizing or sequencing proteins.

Quantitative proteomics is an analytical chemistry technique for identifying and measuring the amount of proteins in a sample. Isobaric labeling is a mass spectrometry technique used in quantitative proteomics, whereby peptides or proteins are labeled with mass tags which are then cleaved at specific linker regions yielding reporter ions of different masses. The mass spectrometer detects these reporter ion signals, thereby providing quantitative information regarding the relative amounts of peptides or proteins in the sample.

Multiplexed proteomics experiments generate complex data structures. The data are multi-leveled (many observations within each sample), unbalanced (different numbers of observations in each sample), and heteroskedastic (variability decreases as signals increase) with a matching structure determined by the co-isolation of ions in each scan. When known quantitative proteomics techniques are used to measure the amount (or “abundance”) of peptides and proteins in a sample, the resulting measurements are often inaccurate or misleading.

It is often desirable to fit statistical models to proteomic data (e.g., to estimate parameters of a statistical model such that the model fits the data). Such models have many practical applications, as described below. However, existing techniques for analyzing proteomic attributes of biological samples introduce significant inaccuracy into the estimates of such parameters, and are generally unable to estimate a large number of such parameters, which limits the practical value of such models. Improved techniques for measuring and analyzing proteomic attributes of biological samples are needed.

### SUMMARY

Disclosed herein, in some aspects, are systems and methods of analyzing multiplexed mass spectrometry proteomics data that reduce estimation error when combining multiple isobaric batches. In some embodiments, said systems and methods account for known sources of variation across batches, including the number and quality of measurements observed from each peptide and/or protein, and thereby help avoid and/or reduce the information loss that occurs when summarizing and normalizing peptide and/or protein abundance in a given sample.

Disclosed herein, in some aspects, is a method for processing multiplexed mass spectrometry proteomics data (“MSPD”) from a plurality of batches, each batch comprising a plurality of samples that each comprise one or more peptides, the method comprising: a) obtaining, from the MSPD, one or both of i) reporter ion intensities or a derivative thereof (“intensities”) and ii) reporter ion signal-to-noise ratios or a derivative thereof (“SNRs”) for each peptide in a given sample, wherein the intensities SNRs correspond to one or more scans performed on the given sample; b) receiving, for each scan of the one or more scans in each sample, corresponding covariate values for one or more covariates; c) for each respective parameter of one or more parameters of a statistical model, performing a computation to estimate the respective parameter, each of the one or more parameters representing an association between i) intensities of at least one respective peptide of the one or more peptides and ii) the covariates, the computation comprising: i) appending a design matrix from the statistical model to incorporate intensities from a bridge sample to allow estimating one or more scan specific nuisance variables, the bridge sample representing a pooled sample from each of the one or more batches; ii) weighting the intensities based on the corresponding SNR; iii) fitting the statistical model to the weighted intensities; and iv) outputting an estimate of the parameter and one or more p-values of one or more hypothesis tests for the parameter.

In some embodiments, the one or more peptides correspond to a protein. In some embodiments, the computation further comprises identifying one or more of the parameters to be estimable based on the intensities and the statistical model, wherein outputting the estimate of the parameter and the one or more p-values corresponds to an estimable parameter.

In some embodiments, the method further comprises identifying any intensities for a respective scan in a given sample that has an intensity less than a threshold, wherein weighting said intensities for each of said identified intensities comprises a down weighted value instead of the corresponding SNR or derivative thereof. In some embodiments, the threshold is a percentage of a total summed signal of intensities in a given batch. In some embodiments, the percentage is at most about 0.5%, 1%, 1.5%, 2%, or 3%.

In some embodiments, the method further comprising removing any outliers identified with the intensities and/or SNR. In some embodiments, the covariate values correspond to the number of parameters of the statistical model. In some embodiments, each covariate comprises a covariate factor, a continuous covariate, and/or a time trend within one or more levels of a factor. In some embodiments, the time trends comprise a linear time trend, a cubic time trend, a quadratic time trend, a circadian time trend, or any combination thereof. In some embodiments, the covariate corresponds to an environmental condition and/or a characteristic of a subject from where a peptide was obtained. In some embodiments, the environmental condition comprises a media type for a sample, a dilution factor for a peptide or the protein, a temperature of the sample, or any combination thereof. In some embodiments, the characteristic of a subject comprises an age of the subject, an ethnicity of the subject, a sex of the subject, a height of the subject, a weight of the subject, a physical attributed of the subject, a medical diagnosis of the subject, the subject being administered a treatment, the subject intaking a medication, a location for the protein, a type of medical condition, a cell type, or any combination thereof. In some embodiments, the location of the targeted protein comprises a tissue of the subject. In some embodiments, the tissue comprises a brain, a lung, a heart, a skin, a liver, a stomach, or any combination thereof.

In some embodiments, the covariate comprises a covariate factor, wherein the covariate values for the covariate factor identifies a number of levels pertaining to the factor. In some embodiments, the covariate comprises a continuous factor, wherein the covariate values for the continuous covariate identifies a numerical value. In some embodiments, the statistical model further comprises a sample identification parameter that distinguishes a plurality of samples based on the same source, so as to account for variance between the plurality of samples. In some embodiments, the sample identification parameter is configured to fit the design matrix and/or the appended design matrix to a longitudinal model. In some embodiments, the statistical model is a multi-level model to account for correlations between intensities of a same sample. In some embodiments, the method further comprising adjusting a p-value of the one or more p-values to account for small sample sizes. In some embodiments, adjusting the p-value comprises using Kenward-Roger corrections. In some embodiments, each scan specific nuisance variable corresponds to a scan to scan variation between two or more batches.

Described herein, in another aspect, is a non-transitory computer readable medium for processing multiplexed mass spectrometry proteomics data (“MSPD”) from a plurality of batches, each batch comprising a plurality of samples that each comprise one or more peptides, the non-transitory computer readable medium comprising instructions that, when executed by a processor, cause the processor to perform operations including: a) receiving, from the MSPD, one or both of i) reporter ion intensities or a derivative thereof (“intensities”) and ii) reporter ion signal-to-noise ratios or a derivative thereof (“SNRs”) for each peptide in a given sample, wherein the intensities SNRs correspond to one or more scans performed on the given sample; b) receiving, for each scan of the one or more scans in each sample, corresponding covariate values for one or more covariates; c) for each respective parameter of one or more parameters of a statistical model, performing a computation to estimate the respective parameter, each of the one or more parameters representing an association between i) intensities of at least one respective peptide of the one or more peptides and ii) the covariates, the computation comprising: i) appending a design matrix from the statistical model to incorporate intensities from a bridge sample to allow estimating one or more scan specific nuisance variables, the bridge sample representing a pooled sample from each of the one or more batches; ii) weighting the intensities based on the corresponding SNR; iii) fitting the statistical model to the weighted intensities; and iv) outputting an estimate of the parameter and one or more p-values of one or more hypothesis tests for the parameter.

In some embodiments, the one or more peptides correspond to a protein. In some embodiments, the operations further includes identifying one or more of the parameters to be estimable based on the intensities and the statistical model, wherein outputting the estimate of the parameter and the one or more p-values corresponds to an estimable parameter.

In some embodiments, the operations further includes identifying any intensities for a respective scan in a given sample that has an intensity less than a threshold, wherein weighting said intensities for each of said identified intensities comprises a down weighted value instead of the corresponding SNR or derivative thereof. In some embodiments, the threshold is a percentage of a total summed signal of intensities in a given batch. In some embodiments, the percentage is at most about 0.5%, 1%, 1.5%, 2%, or 3%.

In some embodiments, the operations further includes removing any outliers identified with the intensities and/or SNR. In some embodiments, the covariate values correspond to the number of parameters of the statistical model. In some embodiments, each covariate comprises a covariate factor, a continuous covariate, and/or a time trend within one or more levels of a factor. In some embodiments, the time trends comprise a linear time trend, a cubic time trend, a quadratic time trend, a circadian time trend, or any combination thereof. In some embodiments, the covariate corresponds to an environmental condition and/or a characteristic of a subject from where a peptide was obtained. In some embodiments, the environmental condition comprises a media type for a sample, a dilution factor for a peptide or the protein, a temperature of the sample, or any combination thereof. In some embodiments, the characteristic of a subject comprises an age of the subject, an ethnicity of the subject, a sex of the subject, a height of the subject, a weight of the subject, a physical attributed of the subject, a medical diagnosis of the subject, the subject being administered a treatment, the subject intaking a medication, a location for the protein, a type of medical condition, a cell type, or any combination thereof. In some embodiments, the location of the targeted protein comprises a tissue of the subject. In some embodiments, the tissue comprises a brain, a lung, a heart, a skin, a liver, a stomach, or any combination thereof.

In some embodiments, the covariate comprises a covariate factor, wherein the covariate values for the covariate factor identifies a number of levels pertaining to the factor. In some embodiments, the covariate comprises a continuous factor, wherein the covariate values for the continuous covariate identifies a numerical value. In some embodiments, the statistical model further comprises a sample identification parameter that distinguishes a plurality of samples based on the same source, so as to account for variance between the plurality of samples. In some embodiments, the sample identification parameter is configured to fit the design matrix and/or the appended design matrix to a longitudinal model. In some embodiments, the statistical model is a multi-level model to account for correlations between intensities of a same sample. In some embodiments, the operations further includes adjusting a p-value of the one or more p-values to account for small sample sizes. In some embodiments, adjusting the p-value comprises using Kenward-Roger corrections. In some embodiments, each scan specific nuisance variable corresponds to a scan to scan variation between two or more batches.

Disclosed herein, in other aspects, is a method for processing multiplexed mass spectrometry proteomics data (“MSPD”) from a one or more batches, each batch comprising a plurality of samples that each comprise one or more peptides, the method comprising: a) receiving, from the MSPD, one or both of i) reporter ion intensities or a derivative thereof (“intensities”) and ii) reporter ion signal-to-noise ratios or a derivative thereof (“SNRs”) for each peptide in a given sample, wherein the intensities SNRs correspond to one or more scans performed on the given sample; b) receiving, for each scan of the one or more scans in each sample, corresponding covariate values for one or more covariates; c) for each respective parameter of one or more parameters of a statistical model, performing a computation to estimate the respective parameter, each of the one or more parameters representing an association between i) intensities of at least one respective peptide of the one or more peptides and ii) the covariates, the computation comprising: i) weighting the intensities based on the corresponding SNR; ii) fitting the statistical model to the weighted intensities; and iii) outputting an estimate of the parameter and one or more p-values of one or more hypothesis tests for the parameter.

In some embodiments, the one or more peptides correspond to a protein. In some embodiments, the computation further comprises identifying one or more of the parameters to be estimable based on the intensities and the statistical model, wherein outputting the estimate of the parameter and the one or more p-values corresponds to an estimable parameter.

In some embodiments, the method further comprises identifying any intensities for a respective scan in a given sample that has an intensity less than a threshold, wherein weighting said intensities for each of said identified intensities comprises a down weighted value instead of the corresponding SNR or derivative thereof. In some embodiments, the threshold is a percentage of a total summed signal of intensities in a given batch. In some embodiments, the percentage is at most about 0.5%, 1%, 1.5%, 2%, or 3%.

In some embodiments, the method further comprising removing any outliers identified with the intensities and/or SNR. In some embodiments, the covariate values correspond to the number of parameters of the statistical model. In some embodiments, each covariate comprises a covariate factor, a continuous covariate, and/or a time trend within one or more levels of a factor. In some embodiments, the time trends comprise a linear time trend, a cubic time trend, a quadratic time trend, a circadian time trend, or any combination thereof. In some embodiments, the covariate corresponds to an environmental condition and/or a characteristic of a subject from where a peptide was obtained. In some embodiments, the environmental condition comprises a media type for a sample, a dilution factor for a peptide or the protein, a temperature of the sample, or any combination thereof. In some embodiments, the characteristic of a subject comprises an age of the subject, an ethnicity of the subject, a sex of the subject, a height of the subject, a weight of the subject, a physical attributed of the subject, a medical diagnosis of the subject, the subject being administered a treatment, the subject intaking a medication, a location for the protein, a type of medical condition, a cell type, or any combination thereof. In some embodiments, the location of the targeted protein comprises a tissue of the subject. In some embodiments, the tissue comprises a brain, a lung, a heart, a skin, a liver, a stomach, or any combination thereof.

In some embodiments, the covariate comprises a covariate factor, wherein the covariate values for the covariate factor identifies a number of levels pertaining to the factor. In some embodiments, the covariate comprises a continuous factor, wherein the covariate values for the continuous covariate identifies a numerical value. In some embodiments, the statistical model further comprises a sample identification parameter that distinguishes a plurality of samples based on the same source, so as to account for variance between the plurality of samples. In some embodiments, the sample identification parameter is configured to fit the design matrix and/or the appended design matrix to a longitudinal model. In some embodiments, the statistical model is a multi-level model to account for correlations between intensities of a same sample. In some embodiments, the method further comprising adjusting a p-value of the one or more p-values to account for small sample sizes. In some embodiments, adjusting the p-value comprises using Kenward-Roger corrections.

Disclosed herein, in other aspects, is a method of measuring amounts of one or more peptides in a plurality of batches, each batch comprising a plurality of samples, each sample comprising one or more labeled peptides, the method comprising: a) performing, with a mass spectrometer, quantitative mass spectroscopy on the plurality of batches, thereby obtaining multiplexed mass spectrometry proteomics data (“MSPD”); b) obtaining, from the MSPD, one or both of i) reporter ion intensities or a derivative thereof (“intensities”) and ii) reporter ion signal-to-noise ratios or a derivative thereof (“SNRs”) for each peptide in a given sample, wherein the intensities SNRs correspond to one or more scans performed on the given sample; c) receiving, for each scan of the one or more scans in each sample, corresponding covariate values for one or more covariates; for each respective parameter of one or more parameters of a statistical model, performing a computation to estimate the respective parameter, each of the one or more parameters representing an association between i) intensities of at least one respective peptide of the one or more peptides and ii) the covariates, the computation comprising: i) appending a design matrix from the statistical model to incorporate intensities from a bridge sample to allow estimating one or more scan specific nuisance variables, the bridge sample representing a pooled sample from each of the one or more batches; ii) weighting the intensities based on the corresponding SNR; iii) fitting the statistical model to the weighted intensities; and iv) estimating a value of the parameter and one or more p-values of one or more hypothesis tests for the parameter; and e) reporting, based on the estimated values of the one or more parameters of the statistical model, the amounts of the one or more peptides in each of the samples.

In some embodiments, the one or more peptides correspond to a protein. In some embodiments, the computation further comprises identifying one or more of the parameters to be estimable based on the intensities and the statistical model, wherein outputting the estimate of the parameter and the one or more p-values corresponds to an estimable parameter.

In some embodiments, the method further comprises identifying any intensities for a respective scan in a given sample that has an intensity less than a threshold, wherein weighting said intensities for each of said identified intensities comprises a down weighted value instead of the corresponding SNR or derivative thereof. In some embodiments, the threshold is a percentage of a total summed signal of intensities in a given batch. In some embodiments, the percentage is at most about 0.5%, 1%, 1.5%, 2%, or 3%.

In some embodiments, the method further comprising removing any outliers identified with the intensities and/or SNR. In some embodiments, the covariate values correspond to the number of parameters of the statistical model. In some embodiments, each covariate comprises a covariate factor, a continuous covariate, and/or a time trend within one or more levels of a factor. In some embodiments, the time trends comprise a linear time trend, a cubic time trend, a quadratic time trend, a circadian time trend, or any combination thereof. In some embodiments, the covariate corresponds to an environmental condition and/or a characteristic of a subject from where a peptide was obtained. In some embodiments, the environmental condition comprises a media type for a sample, a dilution factor for a peptide or the protein, a temperature of the sample, or any combination thereof. In some embodiments, the characteristic of a subject comprises an age of the subject, an ethnicity of the subject, a sex of the subject, a height of the subject, a weight of the subject, a physical attributed of the subject, a medical diagnosis of the subject, the subject being administered a treatment, the subject intaking a medication, a location for the protein, a type of medical condition, a cell type, or any combination thereof. In some embodiments, the location of the targeted protein comprises a tissue of the subject. In some embodiments, the tissue comprises a brain, a lung, a heart, a skin, a liver, a stomach, or any combination thereof.

In some embodiments, the covariate comprises a covariate factor, wherein the covariate values for the covariate factor identifies a number of levels pertaining to the factor. In some embodiments, the covariate comprises a continuous factor, wherein the covariate values for the continuous covariate identifies a numerical value. In some embodiments, the statistical model further comprises a sample identification parameter that distinguishes a plurality of samples based on the same source, so as to account for variance between the plurality of samples. In some embodiments, the sample identification parameter is configured to fit the design matrix and/or the appended design matrix to a longitudinal model. In some embodiments, the statistical model is a multi-level model to account for correlations between intensities of a same sample. In some embodiments, the method further comprising adjusting a p-value of the one or more p-values to account for small sample sizes. In some embodiments, adjusting the p-value comprises using Kenward-Roger corrections. In some embodiments, each scan specific nuisance variable corresponds to a scan to scan variation between two or more batches.

### BRIEF DESCRIPTION OF THE DRAWINGS

The accompanying figures, which are included as part of the present specification, illustrate the presently preferred embodiments and together with the general description given above and the detailed description of the preferred embodiments given below serve to explain and teach the principles described herein.

FIG. 1 depicts a flow chart of a method for processing multiplexed mass spectrometry proteomics data, according to an embodiment herein.

FIG. 2 depicts a flow chart of a computation method for estimating each parameter of a statistical model, according to an embodiment herein.

FIG. 3 depicts an exemplary covariate file identifying covariate values for the corresponding covariates, according to an embodiment herein.

FIG. 4 depicts an exemplary sample file aligning each sample with a set of covariates, according to an embodiment herein.

FIG. 5 depicts a block diagram of an example computer system, in accordance with some embodiments.

FIG. 6 depicts an exemplary work flow for performing a complete case proteomics data analysis with a large class of experimental designs, in accordance with some embodiments.

FIG. 7 provides a depiction of an Interbatch Benchmarking experiment and experimental workflow, according to an embodiment herein.

FIG. 8 provides an exemplary analysis and comparison of batch composition impacts, according to an embodiment herein.

FIG. 9 provides an exemplary comparison of impacts due to signal magnitude and fold-change, according to an embodiment herein.

FIG. 10 provides an exemplary comparison of LOQ analysis for two groups, according to an embodiment herein.

FIG. 11 provides an exemplary comparison using Boxplots for absolute and relative LOQ analysis, according to an embodiment herein.

FIG. 12 provides exemplary comparisons of performance metrics for the Interbatch Benchmarking experience, according to an embodiment herein.

FIG. 13 provides exemplary data comparisons based on the Ratio Expansion Experiment, according to an embodiment herein.

FIG. 14 provides additional exemplary comparisons of performance metrics for the Interbatch Benchmarking experience, according to an embodiment herein.

FIG. 15 provides an exemplary overview and comparison of application of msTrawler software to a 4-plex senescence experiment, according to an embodiment herein.

FIG. 16 provides an exemplary overview and comparison of application of msTrawler software to a 23 TMT batch study, according to an embodiment herein.

FIG. 17 provides exemplary protein summaries from a CPTAC analysis, according to an embodiment herein.

FIG. 18 provides exemplary comparisons for Pearson correlations between mRNA and Protein levels, according to an embodiment herein.

FIG. 19 provides exemplary protein summary statistics from a TKO standard dataset and Pearson correlations, according to an embodiment herein.

FIG. 20 provides exemplary comparisons of signal to noise ratio detection based on rank ordered genes based on Saccharomyces Genome Database data, according to an embodiment herein.

FIG. 21 provides exemplary comparisons of intensity detection based on rank ordered genes based on Saccharomyces Genome Database data, according to an embodiment herein.

FIG. 22 provides exemplary comparisons for signal dependent variations at equal sample size, according to an embodiment herein.

FIG. 23 provides exemplary comparisons of TMT interference scales with total signal, according to an embodiment herein.

FIG. 24 provides exemplary ratio expansion data based on proportion of log₂ deviations, according to an embodiment herein.

FIG. 25 provides exemplary ratio expansion data based on median deviations, according to an embodiment herein.

FIG. 26 provides exemplary comparisons in a normalization method for determining scan to scan variations, according to an embodiment herein.

FIG. 27 provides exemplary comparisons in another normalization method for determining scan to scan variations, according to an embodiment herein.

FIG. 28 provides exemplary comparisons of methodological differences when estimating small changes in intensity detection, according to an embodiment herein.

FIG. 29 provides exemplary comparisons processing time between regular routine and summation based variant, using a system according to an embodiment herein.

While the present disclosure is subject to various modifications and alternative forms, specific embodiments thereof have been shown by way of example in the drawings and will herein be described in detail. The present disclosure should not be understood to be limited to the particular forms disclosed, but on the contrary, the intention is to cover all modifications, equivalents, and alternatives falling within the spirit and scope of the present disclosure.

### DETAILED DESCRIPTION

Motivation for and/or Benefits of Some Embodiments

Multiplexed proteomics experiments, enabled by isobaric labeling, are increasingly popular for quantifying the relative abundance of peptides and proteins between multiple samples. Advantages of this technology versus label-free experiments include the reduction of instrument time per sample, ease of sample fractionation post-labeling which results in high numbers of identifications, fewer missing values than label-free experiments, and high quantitative precision.

Traditionally, most isobaric labelling applications use just a single batch (i.e., one set of co-isolated isobarically labeled samples) and the majority of the data analysis methods for isobaric labeling have focused on single batch analysis. This approach, however, limits the sample size of an experiment to the number of isobaric tags available (e.g., 18 for certain available products).

In a single batch, the same peptides, eluting at the same time, are compared across all samples in a study. When combining multiple batches both the number and the identities of the observed peptides are subject to change, resulting in missing values (sometimes referred to herein as the “missing data problem” or “incomplete data problem”) and a loss of accuracy. However, the challenge of combining multiple batches goes well beyond that of matching up peptides across runs since even signals from exactly the same peptide can vary substantially between batches. In some cases, this may be a result of signals from isobaric labels being very precise measures of relative abundance but only weakly correlated with the absolute abundance of a protein. In some cases, repeat scans from the same peptide demonstrate high variability as key experimental variables change through time. There is also a relative limit-of-quantitation within each scan. These related challenges may be referred to herein as “the measurement quality problem.”

Some systems for proteomic data analysis attempt to address the incomplete data problem by excluding any proteins that are not observed in all batches from the analysis. Other systems attempt to address the incomplete data problem by imputing estimated measurements for proteins in the batches in which those proteins are not observed. Both of these approaches can introduce significant error into the results of the data analysis. In contrast, proteomic data analysis systems that use the techniques described herein can provide a complete case analysis of multi-batch proteomic data, without excluding measurements of proteins that are not observed in all batches, and without imputing values for the “missing” measurements. In some embodiments, a proteomic data analysis system may determine and report the estimability of one or more (e.g., all) model parameters based on the proteomic data being analyzed.

Some systems for proteomic data analysis also attempt to address the measurement quality problem by summing the reporter ion fluxes. This approach simplifies data analysis by first compiling single number summaries for each protein. While subsequent analyses are indeed simpler, the data reduction comes with a substantial loss of information. Single number summaries do not simultaneously convey the number of observations within each sample, the quality of the observations, or the scan level matching structure across co-isolated compounds. Thus, this approach can introduce significant error into the results of the data analysis. In contrast, some embodiments of proteomic data analysis systems described herein use peptide reporter ion counts (e.g., reporter signal-to-noise ratio) to weight observations of peptide reporter ion flux, such that the reporter ion counts function as indicators of the quality of the corresponding reporter ion flux observations. This use of reporter ion counts can be useful not only for accounting for variation in measurement quality across batches, but also for accounting for variation in measurement quality within an individual batch.

In some embodiments, the techniques described herein may be used to fit statistical models (e.g., linear mixed models (LMMs)) to proteomic data. The fitted models may be used to more accurately estimate any suitable proteomic measurements including, without limitation, (1) the rate of change in abundance of a specified protein over time in a subject having a particular medical condition, (2) the relative abundance of a specified protein between subjects with a different medical condition, etc.

The systems and methods described herein may reduce error caused by variation in the number and quality of observations across batches, while allowing the automatic estimation of model parameters even in the presence of uncontrolled missing data. In some embodiments, such estimation of parameters (e.g., via statistical model(s) as described herein) enable measurement of the effects of perturbations on a global proteome. For example, in some embodiments, such estimation of parameters enable more accurate measurement of the effects of a drug or other treatment applied to a subject, the effects of a mutation on a subject's proteome, etc. In some embodiments, such estimation of parameters enable more accurate characterizations of molecular processes such as replicative senescence. In some embodiments, reliability of the detection of disease biomarkers are improved, based on measurement of one or more peptides and/or proteins with respect to certain parameters.

### Terminology

Terms used in the claims and specification are defined as set forth below unless otherwise specified.

The terms “subject” or “patient” are used interchangeably and encompass a cell, tissue, or organism, human or non-human, whether in vivo, ex vivo, or in vitro, male or female.

As used herein, “proteomic data” refers to values (e.g., quantitative values) reported by a spectrometric instrument (e.g., mass spectrometer) pertaining to peptides (e.g., isolated and identified ionized peptides). The proteomic data may include, without limitation, peptide reporter ion fluxes, peptide reporter ion signal-to-noise ratios (SNRs), identifying attributes (e.g., mass-to-charge ratio and/or charge state), etc.

As used herein, unless otherwise specified, “peptide,” “oligopeptide,” and “polypeptide,” are used interchangeably and refer to a series of amino acids covalently linked by amide bonds. A peptide can contain any number of amino acids of two or greater. In some embodiments, a peptide is 2 to 10, 5 to 10, 10 to 15, 10 to 20, 10 to 25, 10 to 30, 10 to 40, 10 to 50, 25 to 50, or 50 to 100 amino acids in length. In some embodiments, a peptide is about 2, 3, 4, 5, 6, 7, 8, 9, 10, 15, 20, 25, 30, 40, 50, or more amino acids in length, but generally 5-35 amino acids long. As used herein, the terms can refer to a single peptide chain covalently linked by amide bonds. The terms can also refer to multiple peptide chains associated by non-covalent interactions, such as ionic contacts, hydrogen bonds, Van der Waals contacts, and hydrophobic contacts. As used herein, the terms include peptides that contain natural and/or unnatural amino acids or have been modified, e.g., by post-translational processing such as signal peptide cleavage, disulfide bond formation, glycosylation (e.g., N-linked glycosylation), protease cleavage and lipid modification (e.g., S-palmitoylation).

In some embodiments, a “peptide” may be a series of amino acids produces by applying a digestion agent to a solution. In some embodiments, the analytical techniques described herein may involve analyzing measurements of individual peptides rather than analyzing aggregated measurements of multiple peptides (e.g., proteins). In some embodiments, a “peptide” may contain relatively fewer amino acids than a “protein.” For example, a peptide may contain fewer than a threshold number of amino acids (e.g., fewer than approximately 50 amino acids, approximately 5 to 50 amino acids, 7 to 50 amino acids, 5 to 35 amino acids, etc.), and a protein main contain more than the threshold number of amino acids (e.g., more than approximately 50 amino acids). In some embodiments, one or more peptides, singularly or collectively correlate with a given protein. In some embodiments, a protein comprises or consists of one or more peptides.

As used herein, unless otherwise specified, “proteomics” refers to the analysis (e.g., quantitative analysis and/or qualitative analysis) of the proteome, the entire complement or fraction of peptides or proteins expressed by a genome, cell, tissue, organism, organ, tissue, body fluid (e.g., plasma, CSF, urine, etc.) extracellular space, organelle, or any combination thereof, including identities, quantities, localization, structures, functions, interactions, and modifications of proteins at any stage, and how these properties vary in space, time, and physiological state. Proteomics encompasses the investigation of the nature of cellular processes through the characterization of defining properties and behaviors of proteins, such as protein expression profiles, post-translational modifications, intracellular localization, protein-protein interactions, protein complexes with a view to space, time, and physiological state. Various methods to study peptides or proteins are known in the art, e.g., immunoassays (ELISA, Western Blotting, Arrays such as SOMAscan or Proximity Extension Assay) or mass spectrometry. Mass spectrometry proteomics techniques include both labelling methods as well as label-free methods. Labelling methods include but are not limited to isobaric tags such as TMT (tandem mass tags).

As used herein, unless otherwise specified, “protein-protein interaction” or “PPI” refers to the contact, typically with high specificity, between two or more proteins, e.g., through electrostatic forces, hydrogen bonding, and/or hydrophobic effect, as it is known in the art. Protein-protein interactions can be characterized as stable or transient and can occur between identical or non-identical chains.

As used herein, unless otherwise specified, “molecule” refers to any molecular entity, including small molecules (e.g., organic compounds), polymers (e.g., nucleic acids), and biologics (e.g., proteins).

As used herein, “medical condition” refers to any suitable medical condition of a subject including, without limitation, edema, hemorrhage, hematoma, ischemia, dehydration, the presence of a tumor, the presence of cancer, the presence of a particular type of cancer, a cardiac health condition, infection, a specific type of infection, brain degeneration, extravasation, internal bleeding, maternal hemorrhage, aging-related diseases etc.

The phrasing and terminology used herein are for the purpose of description and should not be regarded as limiting.

Connections between components or systems within the figures are not intended to be limited to direct connections. Rather, data or signals between these components may be modified, re-formatted, or otherwise changed by intermediary components. Also, additional or fewer connections may be used. The terms “coupled,” “connected,” or “communicatively coupled” shall be understood to include direct connections, indirect connections through one or more intermediary devices, wireless connections, and so forth.

Reference in the specification to “one embodiment,” “preferred embodiment,” “an embodiment,” “some embodiments,” or “embodiments” means that a particular feature, structure, characteristic, or function described in connection with the embodiment is included in at least one embodiment of the invention and may be in more than one embodiment. Also, the appearance of the above-noted phrases in various places in the specification is not necessarily referring to the same embodiment or embodiments.

The use of certain terms in various places in the specification is for illustration purposes only and should not be construed as limiting. A service, function, or resource is not limited to a single service, function, or resource; usage of these terms may refer to a grouping of related services, functions, or resources, which may be distributed or aggregated.

Furthermore, one skilled in the art shall recognize that (1) certain steps may optionally be performed; (2) steps may not be limited to the specific order set forth herein; (3) certain steps may be performed in different orders; and (4) certain steps may be performed simultaneously or concurrently.

The term “approximately”, the phrase “approximately equal to”, and other similar phrases, as used in the specification and the claims (e.g., “X has a value of approximately Y” or “X is approximately equal to Y”), should be understood to mean that one value (X) is within a predetermined range of another value (Y). The predetermined range may be plus or minus 20%, 10%, 5%, 3%, 1%, 0.1%, or less than 0.1%, unless otherwise indicated.

The indefinite articles “a” and “an,” as used in the specification and in the claims, unless clearly indicated to the contrary, should be understood to mean “at least one.” The phrase “and/or,” as used in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements).

As used in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements).

The use of “including,” “comprising,” “having,” “containing,” “involving,” and variations thereof, is meant to encompass the items listed thereafter and additional items.

Use of ordinal terms such as “first,” “second,” “third,” etc., in the claims to modify a claim element does not by itself connote any priority, precedence, or order of one claim element over another or the temporal order in which acts of a method are performed. Ordinal terms are used merely as labels to distinguish one claim element having a certain name from another element having a same name (but for use of the ordinal term), to distinguish the claim elements.

### Proteomic Data Analysis Techniques

Described herein, in some aspects, are systems and methods for processing multiplexed proteomics data from one or more batches, wherein each batch may be comprised of one or more samples, and wherein each sample may include therein one or more peptides. In some cases, each peptide pertains to a given protein. In some embodiments, such processing of the multiplexed proteomics data identifies correlations and other relationships between i) various variables associated with the samples and ii) the resulting proteomic data measurements. For example, where the proteomic data measurements provide a relative abundance of one or more peptides across various samples and/or other batches, such processing may provide correlations and relationships between sample variables, such as characteristics of a subject (e.g., age, height, ethnicity etc. of a subject from where the peptides are obtained), environmental conditions of a sample (e.g., media type, temperature), and/or time trends (e.g., changes in relative abundance over time). When combining multiple batches of isobaric proteomics data these correlations may be negatively impacted by variations in the number and quality of measurements; some embodiments of the techniques described herein address these problems.

In some embodiments, systems and methods disclosed herein incorporate one or more statistical models to estimate said correlations and/or relationships between the sample variables and resulting proteomic data measurements. In some embodiments, such sample variables correspond to one or more parameters of the statistical model. The system described can reduce error caused by variation in the number and quality of observations across batches, while allowing the automatic estimation of these parameters even in the presence of uncontrolled missing data.

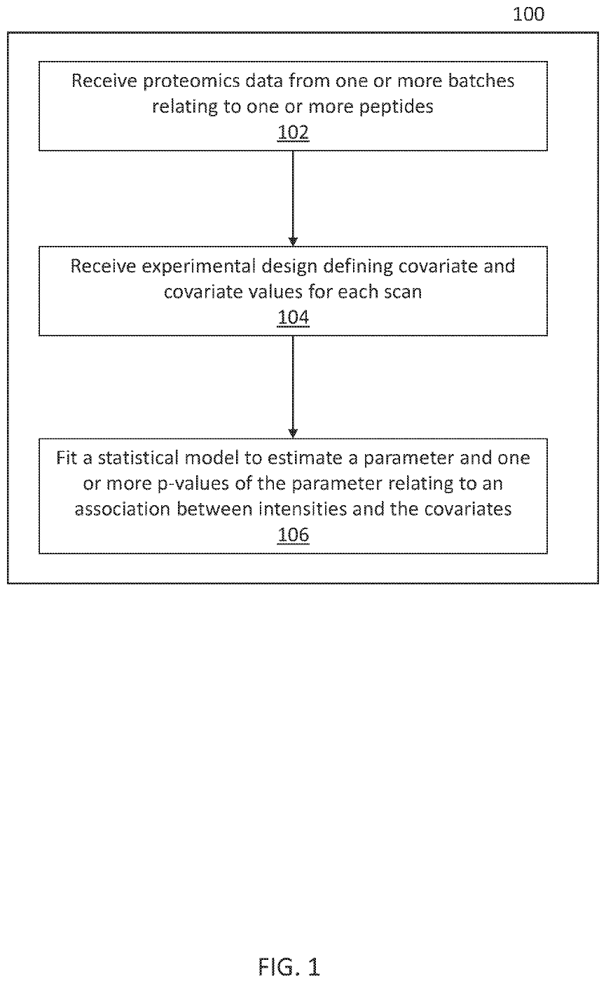

FIG. 1 depicts a flow chart of an exemplary method for processing proteomics data, as described herein. In some embodiments, the method comprises receiving 102 proteomics data from one or more batches of samples. In some embodiments, the method further comprises receiving 104 experimental design information that define covariates and corresponding covariate values (as described herein) for each scan in a mass spectrometry analysis, which correlate to parameters used in the statistical model, as described herein. In some embodiments, the method further comprises fitting 106 the statistical model so as to estimate said parameters, thereby estimating a relationship between the proteomics data and the parameters.

### Proteomics Data

In some embodiments, said proteomics data comprises certain information regarding one or more peptides, and/or one or more proteins, in one or more samples, across one or more batches. In some embodiments, the proteomics data is obtained using any method known in the art. For example, in some embodiments, the proteomics data is obtained using mass spectrometry. In some embodiments, the mass spectrometry comprises isobaric labeling, wherein the one or more peptides in each sample (for example) are tagged with a label (e.g., iTRAQ), and the data measured (relating to released labels) across the mass spectrometry analysis correspond to a quantitative and/or qualitative descriptor of the respective proteomics data. In some embodiments, the mass spectrometry comprises liquid chromatography mass spectrometry.

In some embodiments, as described herein, the proteomics data corresponds to a reporter ion intensity (“intensity”) of a respective isobaric label as detected during the mass spectrometry. In some embodiments, the proteomics data additionally or alternatively comprises a relative intensity, such as for example a reporter ion signal-to-noise ratio (“SNR”). In some embodiments, the proteomics data can be correlated to an abundance of one or more peptides (with or without post-translational modifications), and/or proteins detected in a sample. In some embodiments, the abundance is an absolute abundance (e.g., an absolute amount of peptides detected). In some embodiments, said abundance corresponds to a relative abundance, such as for example, a ratio between an abundance of a given peptide detected to a total abundance of all peptides detected. In some embodiments, the abundance (e.g., absolute or relative) is provided in a logarithmic scale, e.g., a log 2 scale, a log 10 scale, or any other manner as known in the art.

In some embodiments, as described herein, each batch comprises one or more samples. In some embodiments, for each batch, the one or more samples are mixed together prior to the mass spectrometry scans. In some embodiments, any given batch includes at least about 1, 2, 3, 4, 5, 6, 7, 8, 9, 10, 15, 20, 25, 50, 100, 200, 500, or 1000 samples.

In some embodiments, each mass spectrometry analysis for each sample includes one or more scans of the sample. For example, in some embodiments, the mass spectrometry analysis comprises performing multiple scans for the duration of the analysis, thereby detecting multiple measurement data for a given sample, and in some cases, for a given peptide(s). In some embodiments, each detected measurement data corresponds to an observation of the respective peptide (as discussed herein, this may also be an observation of multiple peptides, and/or one or more proteins).

In some embodiments, the proteomics data described herein is based on scans for a single batch or a plurality of batches. In some embodiments, said proteomics data comprises a multiplexed set of information (as described herein).

In some embodiments, the mass spectrometry is performed using hardware that is coupled to and/or spaced apart from hardware use to fit the statistical model. In some embodiments, the raw proteomics data is outputted from the mass spectrometry hardware to a software separate from a software configured to fit the statistical model. In some embodiments, the raw proteomics data is outputted from the mass spectrometry hardware to the same software configured to fit the statistical model. In some cases, wherein the same software is used, different modules within the software are used to obtain the raw proteomics data and fit the statistical model. In some embodiments, the raw proteomics data is initially processed (e.g., before fitting with a statistical model) so as to distinguish the detected data amongst one or more peptides, proteins, and other media material (of which the sample is mixed in). For example, in some cases, an initially processed proteomics data may be partitioned by a given number of specific peptides and/or proteins, such that all other peptides and proteins are not considered for further processing. In some embodiments, a single software having multiple modules is used to initially process the raw proteomics data (including optionally separating out data for a given number of peptides and/or proteins) and fit the statistical model.

Accordingly, in some embodiments, for any system or method herein, the proteomics data (e.g., raw or initially processed) is provided for processing herein.

### Experimental Design Inputs

In some embodiments, a statistical model as described herein incorporates one or more parameters that represent an association between i) an intensity value of an isobaric label detected during a mass spectrometry analysis (for example), which may correspond to an abundance of a peptide and/or protein, and ii) one or more sample variables. In some cases, said sample variables correspond to covariates in statistical terms. In some embodiments, for a given proteomics data analysis, there are one or more types of covariates for which there may exists a relationship or other correlation with the proteomics data, as determined via the statistical model. For example, in some embodiments, the types of covariates comprise any characteristic of a subject, any environmental condition associated with a sample, any conditions relating to the subject, and/or any time trends.

Exemplary characteristics, for example of a subject (e.g., person, animal, other living organism), include type and/or species of an organism, gender, age, height, body mass index (BMI), ethnicity, race, weight, a physical attribute of the subject, a medical diagnosis of the subject, the subject being administered a treatment, the subject intaking a medication, a type of medication being taken, an amount of a medication being taken, or any combination thereof. Other exemplary characteristics may include source location of a sample (e.g., which location within the subject, such as lung, heart, leg, brain, any other tissue of the subject, etc.), cell type, etc. Exemplary conditions include environmental conditions, such as for example, media type of a respective sample, temperature, dilution factor, etc.

In some embodiments, certain covariates are classified as a factor covariate, wherein each factor covariate comprises one or more levels for the respective covariate. For example, for a given characteristic, such as race, the one or more levels may each comprise one of African American, Caucasian, Asian, Hispanic, or Arabic. In some embodiments, each level of a factor covariate would represent a parameter in the statistical model. Accordingly, in the above example for race as the covariate, the statistical model would include 5 parameters for the covariate race (5 parameters corresponding to each type of race specified).

In some embodiments, certain covariates are classified as a continuous covariate, which pertains to a numerical value. For example, characteristics such as age, weight, BMI are described by a specific numerical value. In some embodiments, the parameter would the continuous covariate itself (e.g., age, weight, etc.). In some cases, however, a continuous covariate can be designated as a factor covariate. For example, although age can be a numerical value, it may be categorized by a range of age (e.g., 0-5 years, 6-18 years, 18-45 years, etc.), such that each range of age represents a level for the age covariate.

In some embodiments, time trends refer to a temporal rate of change (for example) of a sample (e.g., intensity readings, abundance), such that the statistical model estimates said time trend. In some embodiments, the time trend is more complex allowing for cubic, quadratic polynomial or circadian trajectories, or any combination thereof. In some embodiments, the time trend may correspond to time trends within levels of a factor covariate, for example temporal responses to a drug treatment may be fit separately to each of many cell lines representing distinct genotypes.

In some embodiments, a sample identification (“sample ID”) is also incorporated with the statistical model so as to distinguish a plurality of samples based on the same source, so as to account for covariance between the plurality of samples. For example, in some cases the sample ID is configured to fit or at least partially fit the statistical model to a longitudinal model.

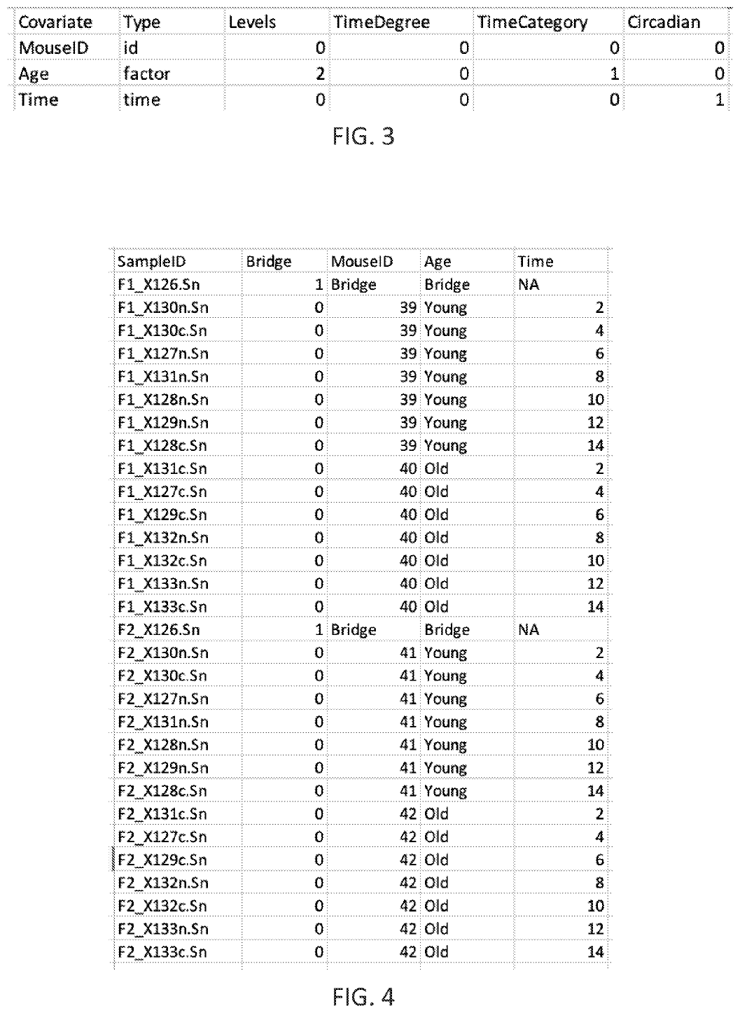

In some embodiments, systems and methods described herein enable fitting a statistical model. Such a model can help provide a visualization of relationships, patterns, and/or correlations between obtained proteomics data, which may correspond to a respective global proteome, and different sample variables and/or variability thereof. In some embodiments, such sample variables (e.g., covariates as described herein) may be specified for a given statistical model, representing variables of interest for which the relative effect on the proteome is desired to be estimated. Accordingly, in some embodiments, the desired covariates and corresponding covariate values (e.g., levels, time trend model) are specified (see FIG. 3 for example, depicting an exemplary covariate file, identifying the covariates and corresponding covariate values, such as 2 levels for age, either young or old). Correspondingly, for each scan in a given sample (of a batch), the detected intensities are matched with the corresponding covariate information (see FIG. 4 for example, depicting an exemplary sample file, wherein the covariates and corresponding parameters are aligned with the respective sample). In some embodiments, such sample file receives the proteomics data information after initial processing (as described herein).

Estimating Parameter and Corresponding p-Value

In some embodiments, the analysis of proteomic data having a large number of covariates and/or covariate levels (e.g. a large multiplexed analysis) leads to difficulty in automatically fitting a statistical model to output an estimation of effect by each parameter, due to the complexity of possible designs. Furthermore, the variability in measurements is a function of the number of ions collected, so in experiments with high variability across samples, some embodiments improve upon prior techniques by accounting for this technical heteroskedasticity. This is true for running mass spectrometry analysis on any number of batches together, such as a single batch, or a plurality of batches.

Similarly, as discussed herein, in some embodiments, the analysis of proteomic data from multiple batches is complicated by properties of the data that vary from batch to batch, including (without limitation) incomplete data (e.g., one or more proteins that are observed in at least one batch and not observed in at least one other batch) and the quality of the measurements (e.g., intensity, sometimes referred to as peptide reporter ion flux, and signal to noise ratio, sometimes referred to as reporter ion count).

Accordingly, in some embodiments, systems and methods described herein enable for such processing of multiplexed mass spectrometry proteomics data, which may include one or more batches. In some embodiments, a statistical model is fitted to account for the variation across each scan and parameters (corresponding to the covariates, as described herein).

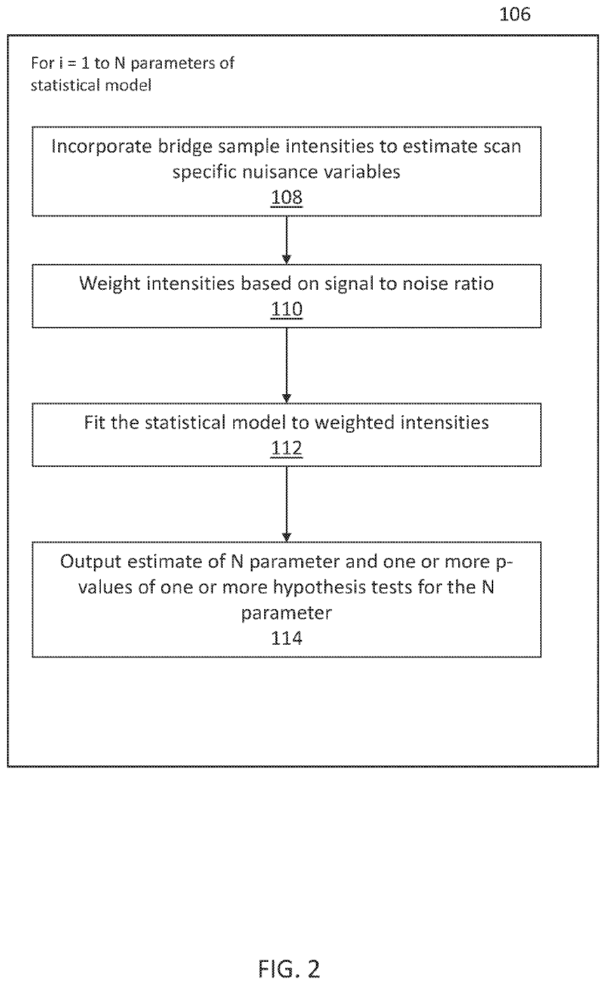

In some embodiments, for each parameter of the statistical model, a computation is performed so as to estimate the respective parameter. Thus, with reference to FIG. 2, in some embodiments, wherein a plurality of parameters N are identified for a given statistical model, a computation I is performed for each parameter, wherein I=1 to N. In some embodiments, the plurality of computations (1 to N) are performed in an iterative manner. In some embodiments, any two or more of the plurality of computations (1 to N) are performed simultaneously. In some embodiments, the plurality of computations (1 to N) are performed in any sequential order and any grouping.

In some embodiments, for each computation, a design matrix aligning the plurality of sample intensities (rows) with the parameter information (columns) is generated for the statistical model. FIG. 2 depicts a flow chart for each computation for each parameter, as described herein.

For multi-batch mass spectrometry analysis, as described herein, in some cases, there exists scan to scan variations between batches that provide variability in the obtained proteomics data. In some embodiments, a bridge sample is utilized to account for such variations, and thereby estimate a scan specific nuisance variable to account for the variation between each batch. In some cases, a bridge sample represents a pooled sample of all the batches, and wherein the bridge sample is included as a sample for each batch. For example, for a given batch, the bridge sample may be tagged with an isobaric label prior to being mixed with the remaining samples so as to be identifiable with the proteomics data.

In some embodiments, the corresponding bridge sample intensities are then incorporated 108 to each design matrix (from each computation) so as to form a respective appended design matrix. Such incorporation into the design matrix includes adding corresponding columns to each design matrix, for which a corresponding nuisance scan specific parameter is thereby included with the statistical model, and which will be considered in the final parameter estimate.

For multi-batch mass spectrometry analysis, as described herein, in some cases, there exists missing proteomic intensity data, such as certain batches not detecting a given peptide. As described herein, in lieu of dropping such intensity data all together or arbitrarily assigning an intensity value (as sometimes performed in existing cases), in some embodiments, systems and methods described herein are configured to determine whether missing intensities have rendered any model parameters inestimable, and adaptively modify the design matrix to enable estimation and inference on all estimable parameters and no others. For example, in some embodiments, based on a set of parameters for a given design matrix, a parameter is estimable if a corresponding column in the design matrix is linearly independent of all other columns in the matrix. Missing values can render certain parameters inestimable (an occurrence sometimes referred to as extrinsic aliasing). Design matrices with no linear dependencies are said to be full rank, and fitting a statistical model will require a full rank design matrix (see Example 13 herein for example). When extrinsic aliasing has occurred we remove linear dependencies from the design matrix until a full rank design matrix has been achieved. Further, we note the set of parameters that were estimable prior to this reduction and only report estimates and inferences from this set. Accordingly, in some embodiments, the parameters and p-values estimated, as described herein, correspond to an estimable parameter (and not an inestimable parameter).

In some embodiments, as described herein, for single batch mass spectrometry analysis, such bridge sample intensity incorporation may not be needed as there will be no batch to batch variation.

With reference to step 110, the intensities for each design matrix (which may be the appended design matrix for multi-batch analyses) are weighted using the corresponding signal to noise ratios (SNR). In some embodiments, such weighting is performed by using any weighting method known in the art, such as for example, weighted least squares.

In some embodiments, one or more intensities from the proteomics data may be significantly lower than other intensity values, though not necessarily zero. In some embodiments, systems and methods described herein are configured to identify any such intensities for a respective scan (e.g., in a given sample) that has an intensity value less than a prescribed threshold. In some embodiments, such prescribed threshold is a percentage of a total summed signal of co-isolated intensities (i.e., intensities in the same single batch), thereby correlating to a relative limit of quantitation. In some embodiments, such percentage is at most about 0.5%, 1%, 1.5%, 2%, 3%, 5%, 10%, or 50%. In some embodiments, so as to reduce an effect estimated by an intensity having a relatively low measurement, the corresponding intensity is weighted to a down weighted value instead of the corresponding SNR or derivative thereof. For example, in some embodiments, said down weighting is based on a percentage of the minimum threshold, such as 25%, 50%, or 75% of the minimum threshold value.

In some embodiments, systems and method described herein further comprise removing outliers from the proteomics data.

In some embodiments, the statistical model comprises a multi-level model to account for correlations between intensities of a same sample. In some embodiments, a system or method described herein further comprises adjusting a p-value of a corresponding parameter to account for deficiencies when using mixed models with small sample sizes. In some embodiments, adjusting the p-value comprises using a Kenward-Roger correction.

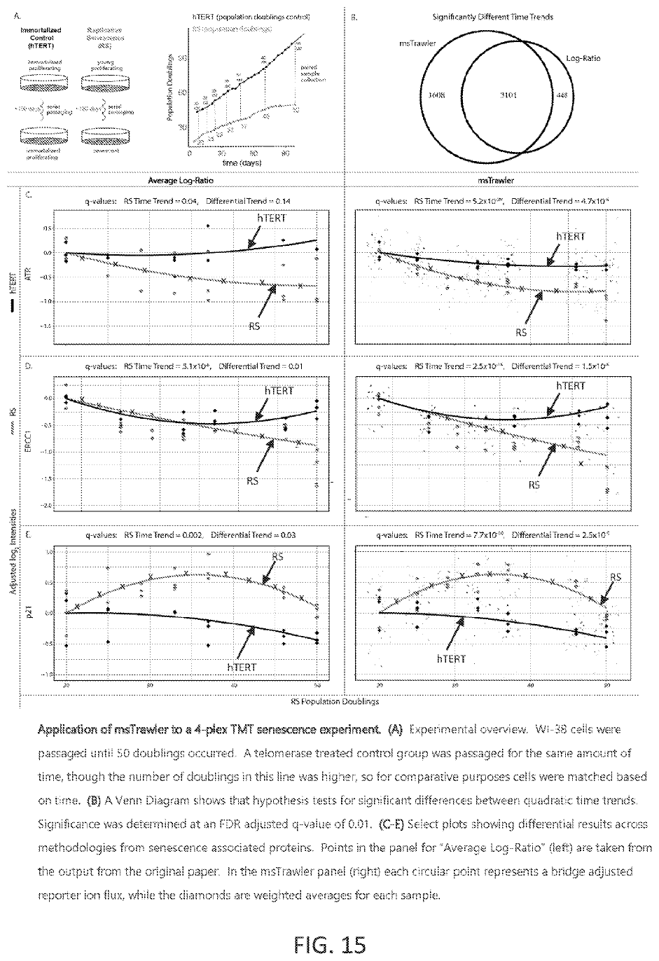

With reference to step 112, the statistical model is then fitted to the weighted intensities, so as to output, for each parameter N, an estimate of the parameter and one or more p-values of one or more hypothesis tests for the N parameter, for example in FIG. 15 we show p-values for a hypothesis of no change in a quadratic time trend along with a test for a differential time trend (is the trend different in the immortalized cell line versus the replicative senescence cell line). Accordingly, the estimates for the plurality of parameters and corresponding p-values provides an estimate of the effect by each parameter on the proteome.

### Some Examples of Computing Devices and Information Handling Systems

In some embodiments, any method described herein, and/or any system described herein, comprises using one or more computing devices/information handling systems/computing systems configured to process the proteomics data. In some embodiments, such one or more computing devices/information handling systems/computing systems execute one or more algorithms to address the complex nature of isobaric proteomics data, as described herein. In some embodiments, any exemplary computing device and/or computing system may be used. For purposes of this disclosure, a computing system may include any instrumentality or aggregate of instrumentalities operable to compute, calculate, determine, classify, process, transmit, receive, retrieve, originate, route, switch, store, display, communicate, manifest, detect, record, reproduce, handle, or utilize any form of information, intelligence, or data for business, scientific, control, or other purposes. For example, a computing system may be a personal computer (e.g., laptop), tablet computer, phablet, personal digital assistant (PDA), smart phone, smart watch, smart package, server (e.g., blade server or rack server), network storage device, or any other suitable device and may vary in size, shape, performance, functionality, and price.

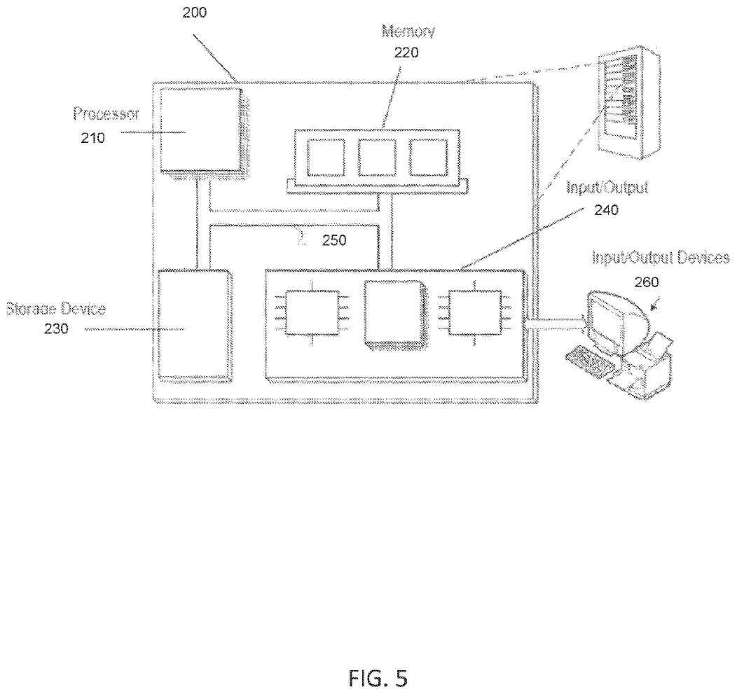

FIG. 5 is a block diagram of an example computer system 200 that may be used in implementing the technology described in this document. General-purpose computers, network appliances, mobile devices, or other electronic systems may also include at least portions of the system 200. The system 200 includes a processor 210, a memory 220, a storage device 230, and an input/output device 240. Each of the components 210, 220, 230, and 240 may be interconnected, for example, using a system bus 250. The processor 210 is capable of processing instructions for execution within the system 200. In some implementations, the processor 210 is a single-threaded processor. In some implementations, the processor 210 is a multi-threaded processor. The processor 210 is capable of processing instructions stored in the memory 220 or on the storage device 230.

The memory 220 stores information within the system 200. In some implementations, the memory 220 is a non-transitory computer-readable medium. In some implementations, the memory 220 is a volatile memory unit. In some implementations, the memory 220 is a non-volatile memory unit.

The storage device 230 is capable of providing mass storage for the system 200. In some implementations, the storage device 230 is a non-transitory computer-readable medium. In various different implementations, the storage device 230 may include, for example, a hard disk device, an optical disk device, a solid-date drive, a flash drive, or some other large capacity storage device. For example, the storage device may store long-term data (e.g., database data, file system data, etc.). The input/output device 240 provides input/output operations for the system 200. In some implementations, the input/output device 240 may include one or more of a network interface device, e.g., an Ethernet card, a serial communication device, e.g., an RS-232 port, and/or a wireless interface device, e.g., an 802.11 card, a wireless modem (e.g., 3G, 4G, 5G, etc.) In some implementations, the input/output device may include driver devices configured to receive input data and send output data to other input/output devices, e.g., keyboard, printer and display devices 260. In some examples, mobile computing devices, mobile communication devices, and other devices may be used.

In some implementations, at least a portion of the approaches described above may be realized by instructions that upon execution cause one or more processing devices to carry out the processes and functions described above. Such instructions may include, for example, interpreted instructions such as script instructions, or executable code, or other instructions stored in a non-transitory computer readable medium. The storage device 230 may be implemented in a distributed way over a network, for example as a server farm or a set of widely distributed servers, or may be implemented in a single computing device.

Although an example processing system has been described in FIG. 5, embodiments of the subject matter, functional operations and processes described in this specification can be implemented in other types of digital electronic circuitry, in tangibly-embodied computer software or firmware, in computer hardware, including the structures disclosed in this specification and their structural equivalents, or in combinations of one or more of them. Embodiments of the subject matter described in this specification can be implemented as one or more computer programs, i.e., one or more modules of computer program instructions encoded on a tangible nonvolatile program carrier for execution by, or to control the operation of, a data processing apparatus. Alternatively or in addition, the program instructions can be encoded on an artificially generated propagated signal, e.g., a machine-generated electrical, optical, or electromagnetic signal that is generated to encode information for transmission to suitable receiver apparatus for execution by a data processing apparatus. The computer storage medium can be a machine-readable storage device, a machine-readable storage substrate, a random or serial access memory device, or a combination of one or more of them.

The term “system” may encompass all kinds of apparatus, devices, and machines for processing data, including by way of example a programmable processor, a computer, or multiple processors or computers. A processing system may include special purpose logic circuitry, e.g., an FPGA (field programmable gate array) or an ASIC (application specific integrated circuit). A processing system may include, in addition to hardware, code that creates an execution environment for the computer program in question, e.g., code that constitutes processor firmware, a protocol stack, a database management system, an operating system, or a combination of one or more of them.

A computer program (which may also be referred to or described as a program, software, a software application, a module, a software module, a script, or code) can be written in any form of programming language, including compiled or interpreted languages, or declarative or procedural languages, and it can be deployed in any form, including as a standalone program or as a module, component, subroutine, or other unit suitable for use in a computing environment. A computer program may, but need not, correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program can be deployed to be executed on one computer or on multiple computers that are located at one site or distributed across multiple sites and interconnected by a communication network.

The processes and logic flows described in this specification can be performed by one or more programmable computers executing one or more computer programs to perform functions by operating on input data and generating output. The processes and logic flows can also be performed by, and apparatus can also be implemented as, special purpose logic circuitry, e.g., an FPGA (field programmable gate array) or an ASIC (application specific integrated circuit).

Computers suitable for the execution of a computer program can include, by way of example, general or special purpose microprocessors or both, or any other kind of central processing unit. Generally, a central processing unit will receive instructions and data from a read-only memory or a random access memory or both. A computer generally includes a central processing unit for performing or executing instructions and one or more memory devices for storing instructions and data. Generally, a computer will also include, or be operatively coupled to receive data from or transfer data to, or both, one or more mass storage devices for storing data, e.g., magnetic disks, magneto optical disks, or optical disks. However, a computer need not have such devices. Moreover, a computer can be embedded in another device, e.g., a mobile telephone, a personal digital assistant (PDA), a mobile audio or video player, a game console, a Global Positioning System (GPS) receiver, or a portable storage device (e.g., a universal serial bus (USB) flash drive), to name just a few.

Computer readable media suitable for storing computer program instructions and data include all forms of nonvolatile memory, media and memory devices, including by way of example semiconductor memory devices, e.g., EPROM, EEPROM, and flash memory devices; magnetic disks, e.g., internal hard disks or removable disks; magneto optical disks; and CD-ROM and DVD-ROM disks. The processor and the memory can be supplemented by, or incorporated in, special purpose logic circuitry.

To provide for interaction with a user, embodiments of the subject matter described in this specification can be implemented on a computer having a display device, e.g., a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input. In addition, a computer can interact with a user by sending documents to and receiving documents from a device that is used by the user; for example, by sending web pages to a web browser on a user's user device in response to requests received from the web browser.

Embodiments of the subject matter described in this specification can be implemented in a computing system that includes a back end component, e.g., a data server, or that includes a middleware component, e.g., an application server, or that includes a front end component, e.g., a client computer having a graphical user interface or a Web browser through which a user can interact with an implementation of the subject matter described in this specification, or any combination of one or more such back end, middleware, or front end components. The components of the system can be interconnected by any form or medium of digital data communication, e.g., a communication network. Examples of communication networks include a local area network (“LAN”) and a wide area network (“WAN”), e.g., the Internet.

The computing system can include clients and servers. A client and server are generally remote from each other and typically interact through a communication network. The relationship of client and server arises by virtue of computer programs running on the respective computers and having a client-server relationship to each other.

While this specification contains many specific implementation details, these should not be construed as limitations on the scope of what may be claimed, but rather as descriptions of features that may be specific to particular embodiments. Certain features that are described in this specification in the context of separate embodiments can also be implemented in combination in a single embodiment. Conversely, various features that are described in the context of a single embodiment can also be implemented in multiple embodiments separately or in any suitable sub-combination. Moreover, although features may be described above as acting in certain combinations and even initially claimed as such, one or more features from a claimed combination can in some cases be excised from the combination, and the claimed combination may be directed to a sub-combination or variation of a sub-combination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the embodiments described above should not be understood as requiring such separation in all embodiments, and it should be understood that the described program components and systems can generally be integrated together in a single software product or packaged into multiple software products.

Various system components may or may not be in physical proximity to one another. For example, input data and/or output data may be remotely transmitted from one physical location to another. In addition, programs that implement various aspects of some embodiments may be accessed from a remote location (e.g., a server) over a network. Such data and/or programs may be conveyed through any of a variety of machine-readable medium including, but not limited to: magnetic media such as hard disks, floppy disks, and magnetic tape; optical media such as CD-ROMs and holographic devices; magneto-optical media; and hardware devices that are specially configured to store or to store and execute program code, such as application specific integrated circuits (ASICs), programmable logic devices (PLDs), flash memory devices, and ROM and RAM devices. Some embodiments may be encoded upon one or more non-transitory, computer-readable media with instructions for one or more processors or processing units to cause steps to be performed. It shall be noted that the one or more non-transitory, computer-readable media shall include volatile and non-volatile memory. It shall also be noted that alternative implementations are possible, including a hardware implementation or a software/hardware implementation. Hardware-implemented functions may be realized using ASIC(s), programmable arrays, digital signal processing circuitry, or the like. Accordingly, the “means” terms in any claims are intended to cover both software and hardware implementations. Similarly, the term “computer-readable medium or media” as used herein includes software and/or hardware having a program of instructions embodied thereon, or a combination thereof. With these implementation alternatives in mind, it is to be understood that the figures and accompanying description provide the functional information one skilled in the art would require to write program code (i.e., software) and/or to fabricate circuits (i.e., hardware) to perform the processing required.

It shall be noted that some embodiments may further relate to computer products with a non-transitory, tangible computer-readable medium that has computer code thereon for performing various computer-implemented operations. The medium and computer code may be those specially designed and constructed for the purposes of the techniques described herein, or they may be of the kind known or available to those having skill in the relevant arts. Examples of tangible, computer-readable media include, but are not limited to: magnetic media such as hard disks, floppy disks, and magnetic tape; optical media such as CD-ROMs and holographic devices; magneto-optical media; and hardware devices that are specially configured to store or to store and execute program code, such as application specific integrated circuits (ASICs), programmable logic devices (PLDs), flash memory devices, and ROM and RAM devices. Examples of computer code include machine code, such as produced by a compiler, and files containing higher level code that is executed by a computer using an interpreter. Some embodiments may be implemented in whole or in part as machine-executable instructions that may be in program modules that are executed by a processing device. Examples of program modules include libraries, programs, routines, objects, components, and data structures. In distributed computing environments, program modules may be physically located in settings that are local, remote, or both.

One skilled in the art will recognize no computing system or programming language is critical to the practice of the techniques described herein. One skilled in the art will also recognize that a number of the elements described above may be physically and/or functionally separated into sub-modules or combined together.

### EXAMPLES

### Example 1—Comparison Between Isobaric and Label Free Mass Spectrometry Proteomics Experiments

One of the distinguishing characteristics of multiplexed proteomics experiments is that ions are only sampled at a single time point during a relatively long chromatographic elution process. In proteomics experiments that do not use multiplexing, analytes are typically quantified using MS1 spectra collected through time (liquid chromatography mass spectrometry LC-MS). A single peptide species will be scanned many times as molecules elute off the column, and the number of target ions analyzed as a function of time will ideally approximate a bell curve. For label-free quantitation, the area under this curve is representative of the total number of target ions that entered the mass spectrometer, which we expect to correlate with the number of peptides present in the solution. In contrast, isobaric experiments are designed to enable comparisons at a single scan from a single point in time on the elution curve and no effort is ever made to measure all ions from a particular sample. Consequently, reporter ion signals from a single peptide can vary substantially, even within a single batch, simply because the scans may reflect the varying quantity of ions available at different points in the elution curve. Across batches, the sampling time relative to the elution curve, as well as the composition of co-eluting samples, are both likely to change, altering the magnitude of each signal.

Single-timepoint ion sampling presents many data analysis challenges, but the strategy also offers unique advantages. While signals vary substantially from scan-to-scan, relative abundance can be estimated without collecting an entire elution curve. Isobaric experiments therefore have an inherent advantage when performed on samples that have been fractionated prior to LC-MS. Peptides that elute into multiple fractions can complicate label-free quantitation methods, but so long as the separation takes an equal proportion of each isobaric label, the separation has limited or no negative impact on reporter ion quantitation. Similarly, variations in elution time, ionization efficiency, systematic drifts in the instrument, and every other source of technical variability that occurs post-labeling should have proportional effects on each isobaric label. Accordingly, we expect accurate ratios within a single scan even when accurate MS1 peak extraction is not possible. Taken to the extreme, targeted isolation techniques have been shown to allow accurate within-scan reporter ion quantitation even when no MS1 peak can be detected at all. The ability to equalize sources of experimental error across isotopes, while simultaneously eliminating the need to accurately measure an elution curve offers technological advantages for both depth and precision relative to MS1-based quantification techniques. However, when combining samples from multiple batches the sources of technical variation are no longer equalized across all samples.

### Example 2—Variation Across Batches and Bridge Sample

An exemplary strategy to address experimental changes across batches (including elution time and batch composition) is to include a “bridge sample” in each set of co-isolated samples. The bridge is typically generated by combining a small percentage of each sample into one pooled sample prior to labeling and subsequently adding equal amounts of the bridge to each batch, creating a reference against which all the other channels can be compared. Bridge channels are typically incorporated into the data analysis through a normalization step at either the scan level, or after aggregating signals into protein summaries. Both strategies provide similar adjustments and work well to improve ratio accuracy, especially when batch composition changes. Bridge normalization, however, alone cannot account for all the key factors that change across batches.

In addition to compositional and temporal effects, measurement quality (ion counts), the amount of interference, and the number of observations per protein can all change across batches. Taken together, the factors that vary across batches result in highly heteroskedastic data. While the bridge channel can keep ratios on target, it offers no help for situations where the amount of information changes dramatically across batches, for example, ten high quality measurements occur in one batch, while only one low quality measurement is made in the next. Accordingly, such indicators of data quality are typically lost during the processes of aggregation and normalization.

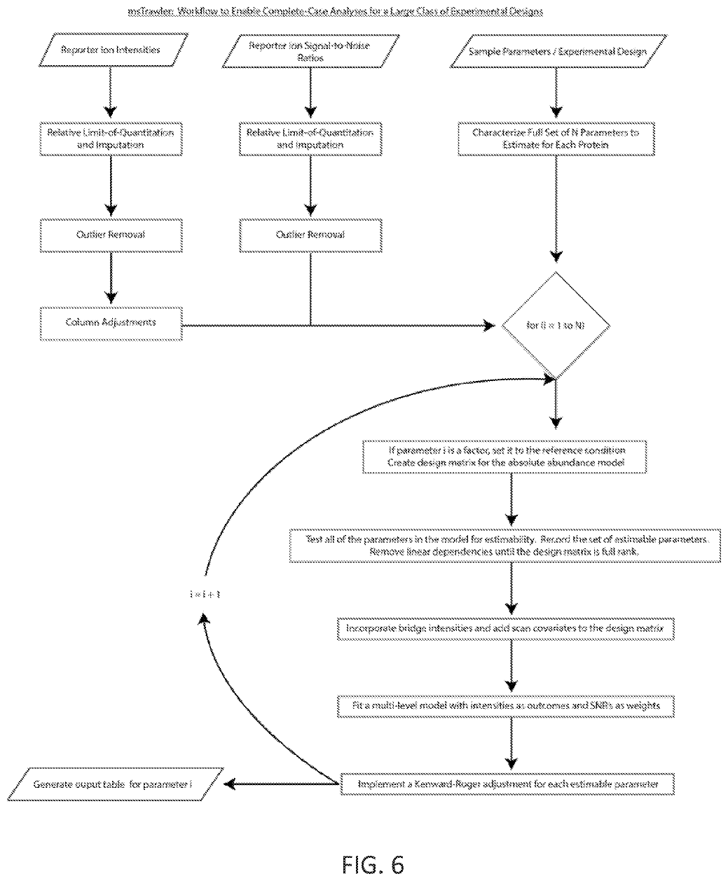

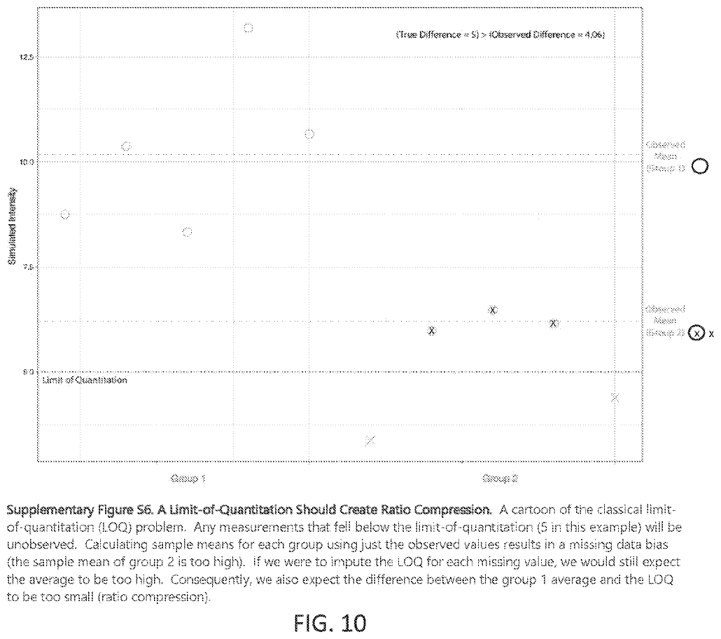

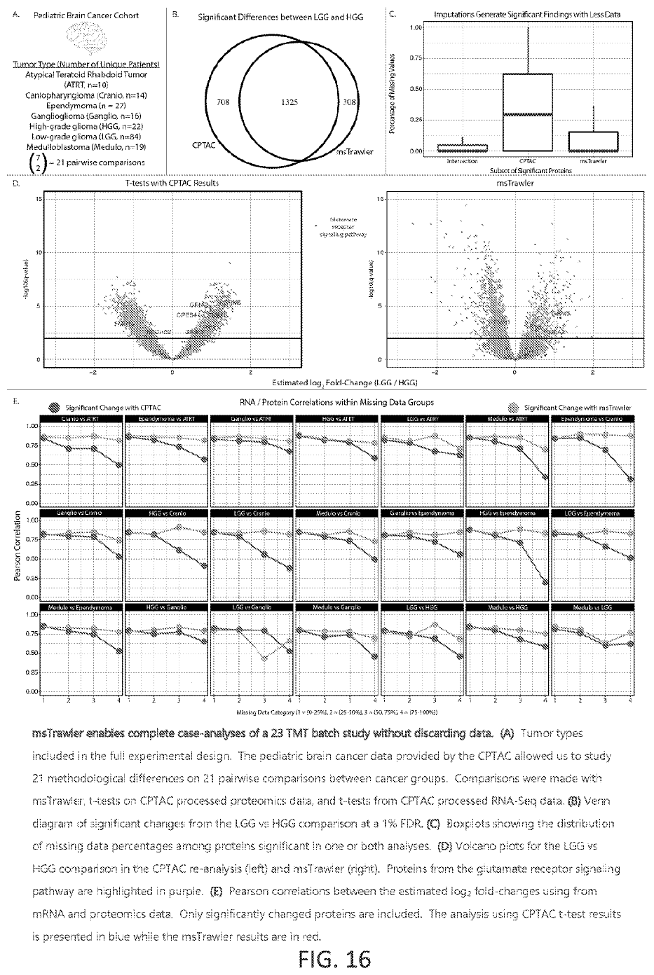

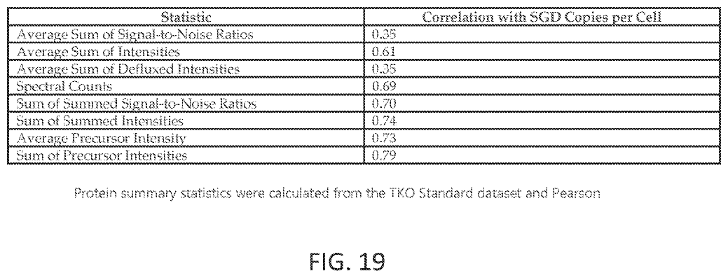

FIG. 6 depicts an exemplary flow chart as used by certain examples to demonstrate processing of multi-batch multiplexed proteomics data. An example of properties varying between batches in a multi-batch proteomics analysis:

| | Feature in System/ | Example or Figure |
|---|---|---|
| Batch varying property | Method described herein | reference |
| Compositional and | Generalized framework | Examples 8, 13, & 14 |
| Temporal Effects | for modeling bridge | FIGS. 8, 19-21, & 26 |
| | samples | |
| Measurement Quality | Flux measurements | Examples 8 & 9 |
| | weighted by | FIGS. 19-22 |
| | corresponding ion counts | |
| Interference and the | A relative limit-of- | Example 11 |
| Limit-of-Quantitation | quantitation (LOQ) is | FIGS. 10, 11, 13, and |
| | established for each | 23-25 |
| | scan. Observations below | |
| | LOQ are imputed and | |
| | down-weighted | |
| Sample Size | Multi-level modeling | Example 14 |
| | with a Kenward-Roger | |
| | correction | |
| Unobserved Proteins | Complete case analyses | FIGS. 6, 16, & 18 |
| | enabled by | |
| | parameterizing, checking | |
| | estimability and | |
| | reporting results | |
| | separately for each | |
| | parameter | |

### Example 3—Interbatch Benchmarking Experiment

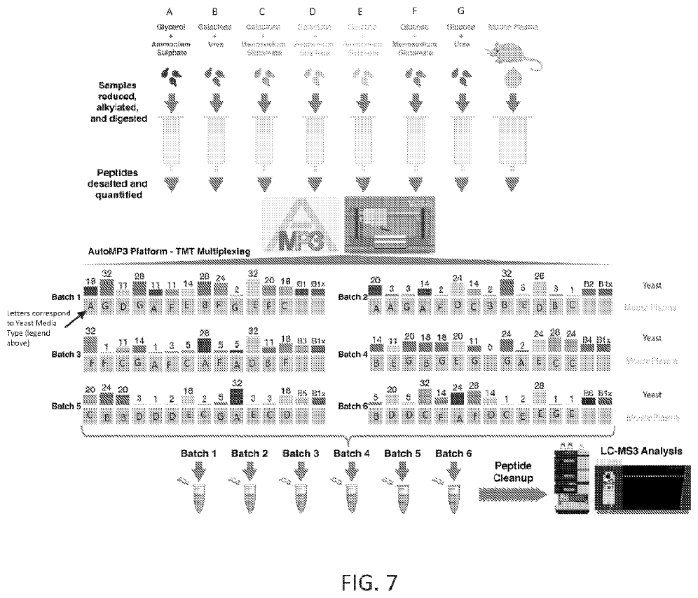

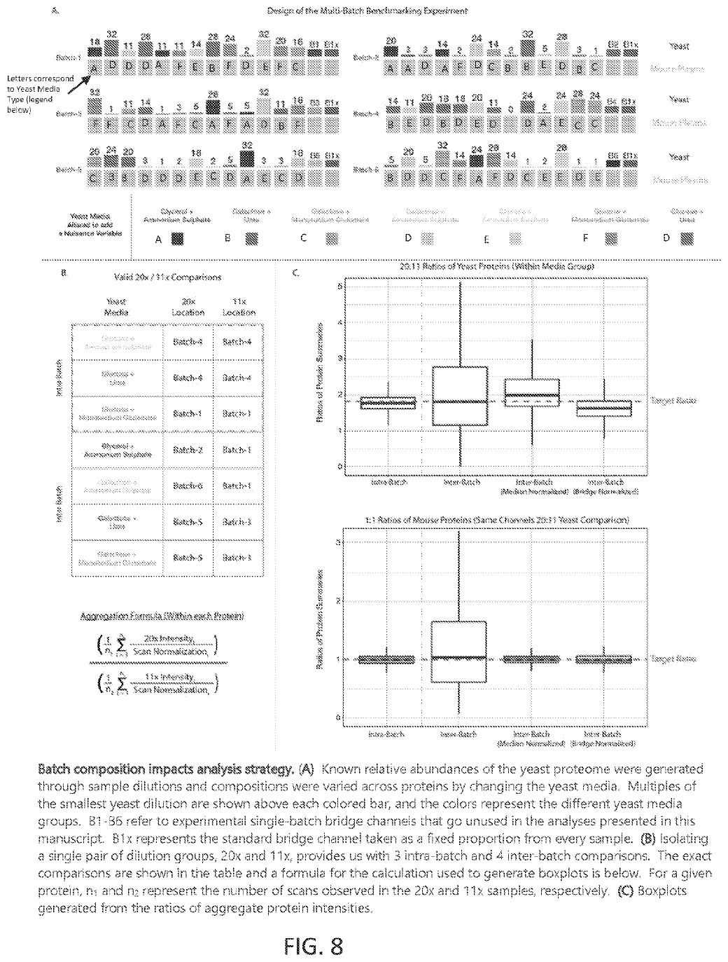

FIG. 7 depicts a multi-batch benchmarking dataset. Samples were prepared using an AutoMP3 method. Mouse plasma was mixed at 1:1 ratios throughout the experiment, while yeast lysate (grown in 7 media types) were diluted at known relative concentrations throughout the 6 batches. B1x is a bridge channel comprised of equal parts from sample, while B1-B6 were experimental localized bridge channels (just mixtures from each batch) that were unused in this study. Samples were analyzed with LC-MS3 on an Orbitrap Eclipse mass spectrometer.

The design is based on a standard two-species dilution model. Mouse plasma was used to create a constant background of potentially interfering peptides at 1:1 ratios. Into this background, various known ratios of yeast cultures were added. For the purpose of investigating how best to combine isobaric batches the experiment was designed with two main goals. First, multiple batches of data containing a wide range of known changes were needed, with some large enough to test the dynamic range of our instrument, and others small enough to probe our capacity for detecting small perturbations. Second, a wide variety of batch compositions to better reflect the full set of patterns that might be observed when studying a random assortment of genetically diverse samples was also needed. To this end, yeast proteomes were diluted at eleven different levels of known changes with a maximum dilution of 1/32 by the use of an automated liquid handler. To generate a diversity of batch compositions across the proteome, yeast was cultured in various carbon and nitrogen source combinations known to substantially alter the yeast proteome. Both media groups and dilution levels were randomly assigned throughout six batches of isobaric-labeled samples.

The importance of batch composition is apparent based on the ratios from the 20× versus 11× comparison, which provides three intra-batch and four inter-batch comparisons (FIG. 8B). Following some basic pre-processing (e.g., see Example 16), boxplots were generated from the ratios of averaged flux measurements with and without various normalization strategies (FIG. 8C). As expected, the inter-batch comparisons, unadjusted for variations across the batches, show higher variability than the corresponding intra-batch comparisons. When comparing methods that attempt to adjust for inter-batch variation (e.g. dividing each scan by the median flux, or by the bridge flux) it was observed the importance of batch composition. For mouse proteins, which never changed in abundance, both normalization strategies have similar boxplots with the median normalization producing slightly less-variable ratios. However, the yeast peptides, which have different compositions in each batch, show a loss of accuracy and precision when taking ratios across batches (FIG. 8C). The increased variation for median normalization was predictable since the median sample comes from different media and dilution groups in each batch. Accordingly, the relationship between the median sample and each target sample also changes across batches. A general discussion about the experimental design and methods to account for inter-batch variation is provided in Example 13.

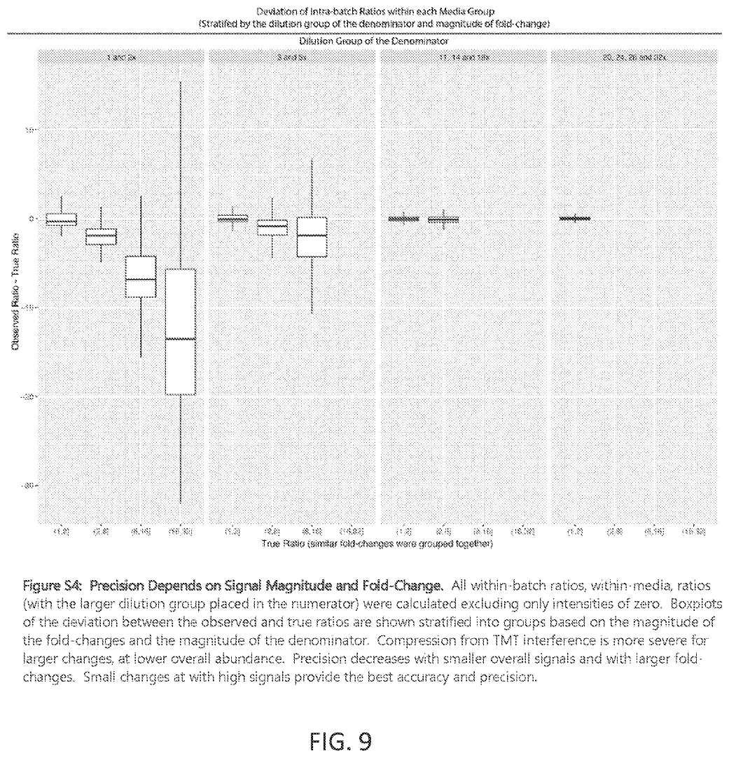

One potential cause of the methodological differences observed across species is the variation in signal magnitude that occurs only in yeast proteins. Looking beyond the 20× to 11× comparison, the full set of deviations between within-batch ratios and their known dilution ratios was explored (FIG. 9). As expected, the larger fold-changes are off target, revealing compression driven by isobaric labeling interference. The precision of all fold-changes was strongly determined by signal magnitude. A closer exploration of the properties of low-signal measurements (Example 9) revealed a consistent relationship between ion counts and signal variance. Above a small threshold, the relationship is highly consistent across the full range of observed ion counts, suggesting that weighted regression models could adequately account for technical variability. However, at the low end of the range, the error spikes up dramatically, suggesting a non-zero limit at which the signals become unreliable.

### Example 4—Relative Limit-of-Quantitation (LOQ)

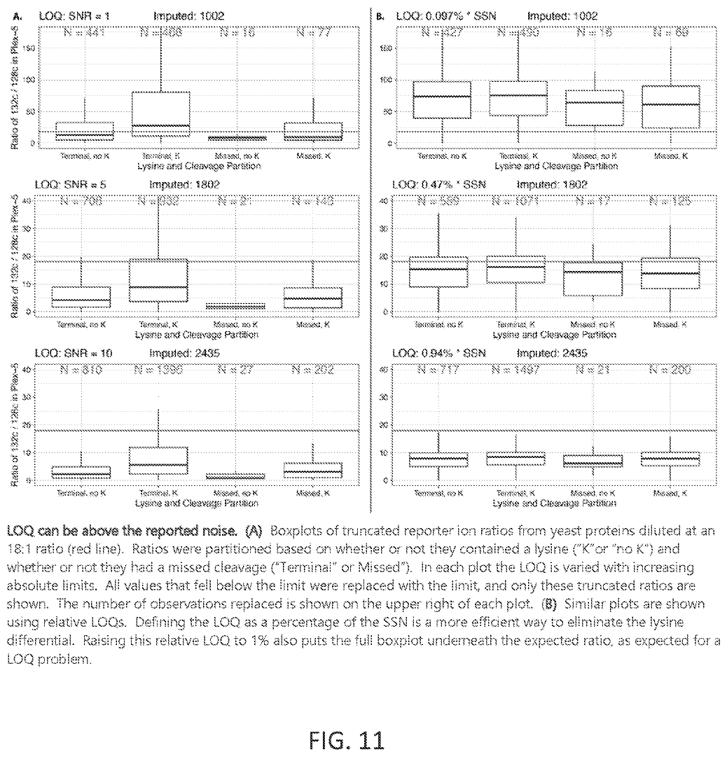

A limit-of-quantitation (LOQ) may be present in cases where a target analyte has no measured flux, but it can also be the case that the analyte of interest will not be present while signals from interfering peptides are still observed. A detailed exploration of the LOQ revealed numerous undesirable properties in the data that occur when signals are low, but non-zero (Example 11). Ratios based on these measurements are often too large, defying expectations of compressed ratios due to both isobaric labeling-interference and the imposition of a LOQ (FIGS. 10-11). A follow-up experiment (Ratio Expansion Experiment) suggested that one of the main causes of this unexpected ratio expansion was the application of a correction for isobaric label (e.g., tandem mass tag (TMT)) isotopic impurities to relatively small signals. It was therefore determined that a more efficient way (preserving more of the data) to identify these problematic cases was to use a relative (proportion of signal) rather than an absolute (ion count) LOQ.

As discussed herein, MsTrawler comprises software configure to receive the covariate data, proteomics data, and fit a statistical model. MsTrawler was built to account for all the above properties of isobaric proteomics data. A method for incorporating bridge channels into arbitrary experimental designs can be used to account for scan-to-scan variation (Example 14). Ion counts are used to implement weighted linear models to account for heteroskedasticity (Example 9, 14). A relative LOQ is implemented to identify unreliable low measurements and these values are then simultaneously imputed at half the LOQ and downweighted to ensure they are not given as much emphasis as actual observations (Example 11). It is important to note that this imputation is only used to resolve limit-of-quantitation issues that occur when low signals have been co-isolated with other samples that are observed above the limit. The imputation is not used to address the far more difficult problem of situations where entire peptides and proteins are unobserved in one or more batches. This latter missing data problem is addressed with the iterative workflow in our software that allows us to adaptatively assess what parameters are estimable based on the pattern of missing data (FIG. 6).

### Example 5: MsTrawler Uses More Data and Increases the Power to Detect Small Changes

Known changes across dilution groups were used to explore method performance. While a system disclosed herein using a respective software (“MsTrawler”) estimates changes directly from reporter ion signals, standard approaches in the field depend on first summarizing protein abundance in each sample and subsequently fitting the protein summaries to statistical models. As used herein, the MsTrawler may correspond to msTrawler software. To compare the effects of summarizing proteins in different ways, four approaches were explored (see Table below) and then apply the same statistical model to estimate changes between dilution groups.

| Summarization | |
|---|---|
| Method | Description |
| MsTrawler | After implementing the relative LOQ and column |
| (2-steps) | adjustments, MsTrawler creates summaries by estimating |
| | the average weighted log-ratio between the bridge and |
| | each sample. |
| LR | Log₂ ratios of every flux, relative to the bridge, are |
| | calculated as a scan-level normalization step. Then |
| | linear models are fit to obtain an average log-ratio for |
| | each sample. |
| SUM | Peptide signals from the same protein are summed |
| | together in each sample. Summed signals are then log₂ |
| | transformed and the corresponding bridge summary is |
| | subtracted from each sample summary (protein level |
| | bridge normalization). |
| cSUM | Identical to SUM, except the bridge normalized |
| | summaries are subsequently centered within each batch |
| | (subtract the batch mean from each value). |

Results from these four approaches were then used as observations in the model zᵢⱼ=β₀+γᵢ+δⱼ+ϵᵢⱼ where zᵢⱼ is a protein summary for dilution group i and media condition j. All the other parameter definitions are identical to those defined in the MsTrawler model (β₀, is the expected signal in the reference category, γᵢ is the difference between dilution level i and the reference dilution, and δⱼ is the difference between the reference media and media group j, details in Example 14). In the following analyses the reference group is the 32× dilution and the accuracy of estimates for γᵢ and the ability to detect changes with the hypothesis test H₀: γᵢ=0 were evaluated.

All analyses were performed on both the data with a 200 SSN filter (1,272 proteins quantified with MsTrawler) and the minimally filtered dataset with an SSN filter of 20 (1,389 proteins quantified with MsTrawler). Due to varying approaches for handling missing intensities, each method returns estimates and p-values for slightly different sets of comparisons. To prevent conflating methodological advantages with differences in the protein changes being estimated, methods only on the subset of proteins with p-values reported for all methods were compared.

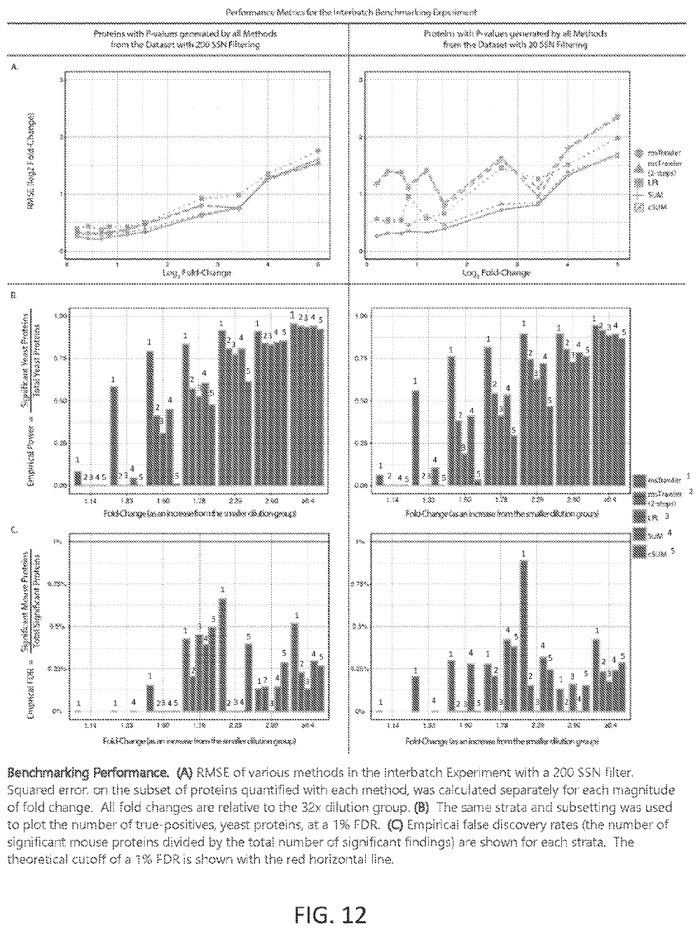

Exploring the accuracy of changes across dilution groups, in the 200 SSN data, it was observed that MsTrawler slightly reduces RMSE for smaller changes (FIG. 12A) and that all methods lost accuracy for the larger changes. The methodological advantage grows considerably when using the data that had been filtered with a 20 SSN cutoff. Calculating RMSE, without stratifying by the magnitude of the change, shows that relaxing the filter from 200 to 20 SSN increases error with SUM by 119% (0.64 to 1.41). The negative effect from including more low-signal scans is mitigated by MsTrawler, with the RMSE only increasing by 12% (0.58 to 0.65). While this showed that efforts to control for low-signal variation improve fold-change estimation, it was anticipated that the largest gains should occur through a better understanding of error and statistical inference.

At a Benjamini-Hochberg adjusted p-value cutoff of 0.01, the MsTrawler model increases the power to detect changes relative to all other methods examined (FIG. 12B). Counting true positives across all dilution groups MsTrawler detects 6,482 and 6,536 yeast proteins in the 200 and 20 SSN datasets, respectively. This is achieved while maintaining an empirical FDR (detected mouse proteins/total detected proteins) less than the theoretical limit of 1% (FIG. 12C). The second most powerful method, SUM, detects 5,302 and 5,114 changes on the 200 and 20 SSN datasets, respectively. Adding the low-signal scans decreases the number of true-positives with SUM, but for MsTrawler the total number of discoveries increases despite the higher RMSE. Overall, the best result from MsTrawler detected 22% more changes than the best result for SUM.

Most of the observed gains come from improved power to detect sub-two-fold changes. For larger changes, the methodological differences are diminished, and we collapsed all the changes greater than 6.4 into a single category since each of these large groups showed approximately equivalent results. A notable advantage here is the stark difference in power between MsTrawler and MsTrawler (2-steps). The only difference between these methods is that MsTrawler directly estimates changes from reporter ion intensities, while MsTrawler (2-steps) first creates summaries of the average ratio to the bridge in each sample, and estimates changes in a subsequent model. This shows that, when estimating changes in dilution groups from bridge normalized protein summaries, the models are no longer able to incorporate information regarding the number, quality or variability observed in the reporter ion fluxes.

Another interesting observation is that the power to detect changes less than or equal to 60% almost completely disappears for cSUM. This method is equivalent to SUM, except for the addition of a seemingly innocuous batch normalization. This normalization could follow from an assumption that the average bridge to sample ratios, in each batch, are equivalent. In The Interbatch Experiment, this assumption is verifiably false, and the consequence is a notable loss of power.

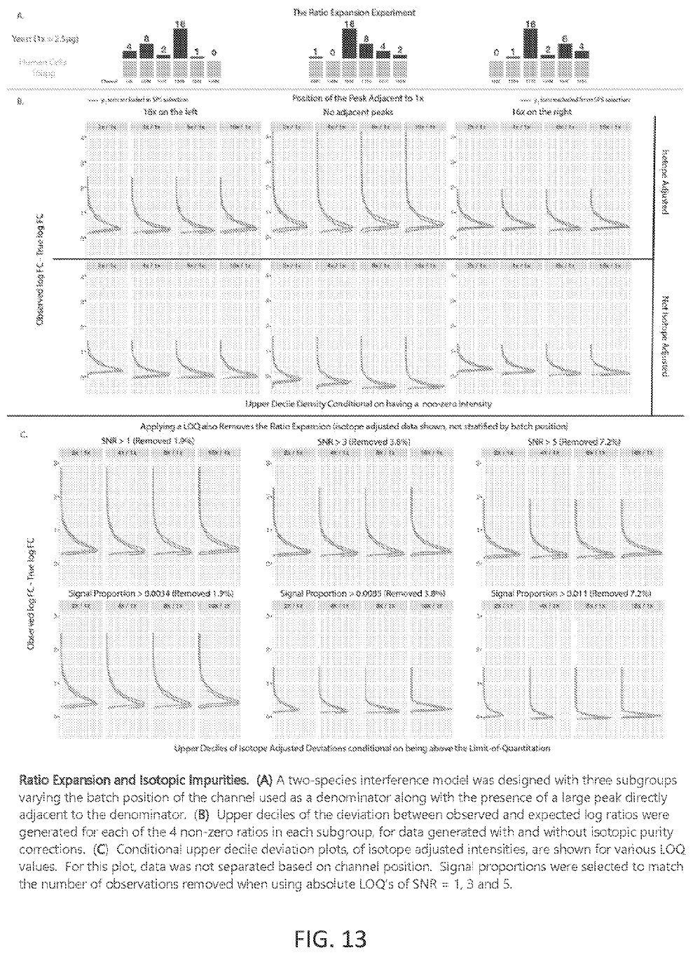

To visualize the sources of the increased power of msTrawler, the data with a 200 SSN filter was evaluated, for which two proteins were highlighted: Inorganic pyrophosphatase (IPP1) (FIG. 13A), which is the most significantly changed protein (msTrawler q-value of 0.0002) in the 1.14 fold-change category and saccharopine dehydrogenase (LYS1) (FIG. 13B), the most significant 33% increase with msTrawler, that was not significant with other methods. The methods largely agree on the estimates of each change, within each media type. In both examples the main difference appears to be to that msTrawler down-weighted the least consistent pairs of observations, creating more confidence in the observed pattern.

An additional set of comparisons adds more evidence to the interpretation of the results. The SSN 20 dataset was modified for compatibility with MSstats-TMT10 by removing all but the most abundant of each repeat peptide. With this dataset we compared msTrawler to (i) msTrawler (SUM) which is an adaptation based on aggregated fluxes and ion counts (Example 14), (ii) msTrawler (no bridge) which is an adaptation that uses row normalization instead of a bridge channel, and (iii) two variations on modeling the summaries from MSstats-TMT—one using the linear model described above (MSstats-lm) and another that additionally includes a random intercept for each batch (MSstats-lmer), which is more consistent with their preferred approach for single factor models. These comparisons show that both RMSE and empirical power were harmed by the row normalization strategy, as should be expected for unbalanced designs.

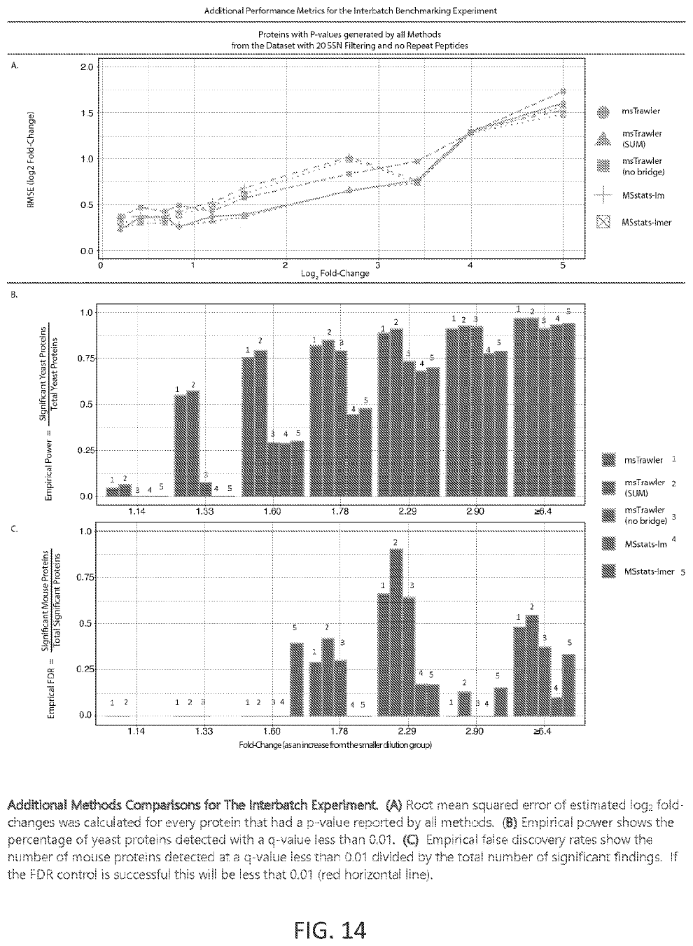

Like the prior results from the SUM and LR methods, both variants of the MSstats-TMT analysis demonstrated a substantial loss of power to detect smaller fold changes (FIG. 14). It is interesting to note that the msTrawler (SUM) preserved and even slightly improved the power to detect small changes. This suggests that the most consequential aspect of our algorithms is the ability to preserve information about the number and quality of peptides across batches. Sums of peptide fluxes increase with both the number and magnitude of each signal. It is only through bridge or scan normalization that this information is typically lost (where high signals become centered near zero).

The gains observed using msTrawler for The Interbatch Experiment suggest that there is a substantial improvement in the ability to detect small changes and reduce error when combining multiple batches. While this experiment lacked many complexities caused by biological variation, the sources of technical variation studied are present in all isobaric proteomics experiments. Consequently, it was predicted that similar gains could be achieved when re-analyzing previously published experiments.

### Example 6—Re-Analyzing a Replicative Senescence Proteome Profiling Time Course Increased the Number of Significantly Changing Proteins by 33%

The system described herein, using a software (“msTrawler”) was evaluated by re-analyzing a 4-batch Tandem Mass Tag (“TMT”) experiment to identify molecular changes in human primary fibroblasts (WI-38 cell line) that occur during the transition to replicative senescence (FIG. 15A). The proteome in the line of cells expected to reach replicative senescence (RS) was repeatedly sampled and compared against an immortalized control treated with telomerase (hTERT). The experiment revealed that a sizeable portion of the proteome changes with cellular age with many trends beginning far before reaching the Hayflick limit of around 50 passages (the point at which human cells, in culture, stop dividing). The published analysis of this dataset included a test for a linear trend through time based on log-ratio protein summaries of the RS line without any formal comparison against the control. Since these summaries contained no information about the number, quality or variation of the peptides within each sample, it was expected that the new approach could reveal new insights into the data by controlling these sources of error.

msTrawler allowed to test for differences in the quadratic time trends between the hTERT and RS cell lines directly from the peptide level observations. The same test of differential quadratic trends was performed using protein summaries from the original study as outcomes in the model (See Example 13 for details). Consistent with the improved power seen in the benchmarking analysis, at a 1% FDR, msTrawler detected an additional 1,160 differential time trends, a 33% increase (FIG. 15B). Unlike the benchmarking experiment, it is unclear how many of these were correct. However, a close look at individual results known to play a role in replicative senescence is encouraging.

Within the GO category for Replicative Senescence, the Log-Ratio method and msTrawler detect 3 and 5 differential time trends, respectively. The two additional potential discoveries were serine/threonine-protein kinase (ATR) and DNA excision repair protein (ERCC1). Additionally, it was observed that a commonly used marker for senescence (which is not included in the GO category) cyclin-dependent kinase inhibitor 1 (p21) also shows different results based on methodology. Curves for these proteins (Example 13), were adjusted to start at zero and were plotted for each method (FIGS. 15C-E). Protein summaries at each timepoint were overlaid for the Log-Ratio plot (left) and weighted averages of adjusted reporter ion intensities (as described in the previous section) are shown in the msTrawler panel (right).

With reference to FIG. 15C, using the Log-Ratio approach, protein summaries from 3 out of 4 of the batches show a consistent decreasing trend. However, all the samples from the other batch have 1-1 ratios, resulting in high residuals and a high q-value. Looking at the scan level data, it was observed that this “outlier” batch contained 12 scans from the ATR protein, but only 4 of them met the quality control filters used in the original data analysis. Ironically, in this case it was the high signal peptides that were inconsistent with the other replicates (which is what was expected if there were many interfering ions or if we had missed identifications). msTrawler utilized all of the available data and found a consistent pattern, that was not dominated by the outlier scan.

ERCC1 (FIG. 15D) showed similar results in both methodologies but the highly significant finding in msTrawler was just above the cutoff when using the Log-Ratio approach. As was observed repeatedly in the benchmarking experiment, the ability to incorporate the peptide level sample size and variance into the analysis often provides more power to detect differences.

The final protein highlighted from this analysis is the traditional marker of senescence, p21 (FIG. 15E). The observed analysis shows the abundance rose ˜50% with a peak near the 35th population doubling (PDL35), and then returned to near baseline levels at PDL50. The same pattern was clearly observed in the previously published Western Blots from this experiment, while the RNA-Seq data showed high levels starting around PDL29 that jumped higher still at PDL50. Using msTrawler, the difference in time trends was highly significant, while the previous methodology produced a borderline result just above our threshold. The additional confidence provided by msTrawler allowed to move away from asking whether or not the observed trend is just noise, to instead focus on the much more interesting question of why a traditional marker for senescence was only increased during the pre-senescent transition. Interestingly, a recent publication showed a therapeutic benefit to targeting and clearing senescent cells with high levels of p21 transcripts but found no benefit when targeting cells with high levels of another common senescence marker, cyclin-dependent kinase inhibitor 2A (p16). If translational regulation is altering the flux of p21 transcripts, then it is plausible that the senolytic therapy achieved its benefits by clearing pre-senescent rather than fully senescent cells.

### Example 7—Application of msTrawler to CPTAC Data Comprising 23 TMT Batches Highlights the Importance of Missing Data Handling Strategies

To evaluate the impact of the system modeling described herein on a large-scale proteomics experiment, data collected from 218 pediatric brain tumors, comprised of seven histologies, with 12 fractions from each sample was re-analyzed. The experiment was performed by the Clinical Proteomic Tumor Analysis Consortium (CPTAC) and the resulting data provide an invaluable resource for exploring brain cancer at the molecular level. Having seven cancer types randomly assigned across 23 batches resulted in data with high variability across samples and many missing values, presenting numerous computational challenges associated with small sample sizes and non-identifiable parameters.

With the intention of presenting a simple comparative analysis, the CPTAC results (processed protein summaries for each sample) was downloaded from the online resource at http://pbt.cptac-data-view.org/and performed pairwise t-tests across histology groups using both proteomics and RNA-Seq data (FIG. 16A). Files were also processed with the msTrawler pipeline. The subset of overlapping proteins (quantified in CPTAC RNA-Seq, CPTAC proteomics and msTrawler) was subsequently compared with an emphasis on low-versus high-grade glioma, selected for simplicity of interpretation.

In contrast with the previous two analyses, fewer significant differences were reported with msTrawler than with t-tests on the CPTAC results (FIG. 16B). Proteins were partitioned into groups based on which methodology found them to be significantly altered. Using pre-imputed CPTAC data (provided by the authors) to determine the percentage of missing values for each protein, we observe that proteins that are uniquely significant in the CPTAC t-test analysis are more likely to have high percentages of missing values (FIG. 16C). Missing values are a substantial source of differences in the methodologies since the CPTAC analysis relied on the DreamAI algorithm to impute values wherever they were missing, while the msTrawler workflow is designed to enable complete-case analyses whenever the statistical parameters in question are estimable (as described herein).

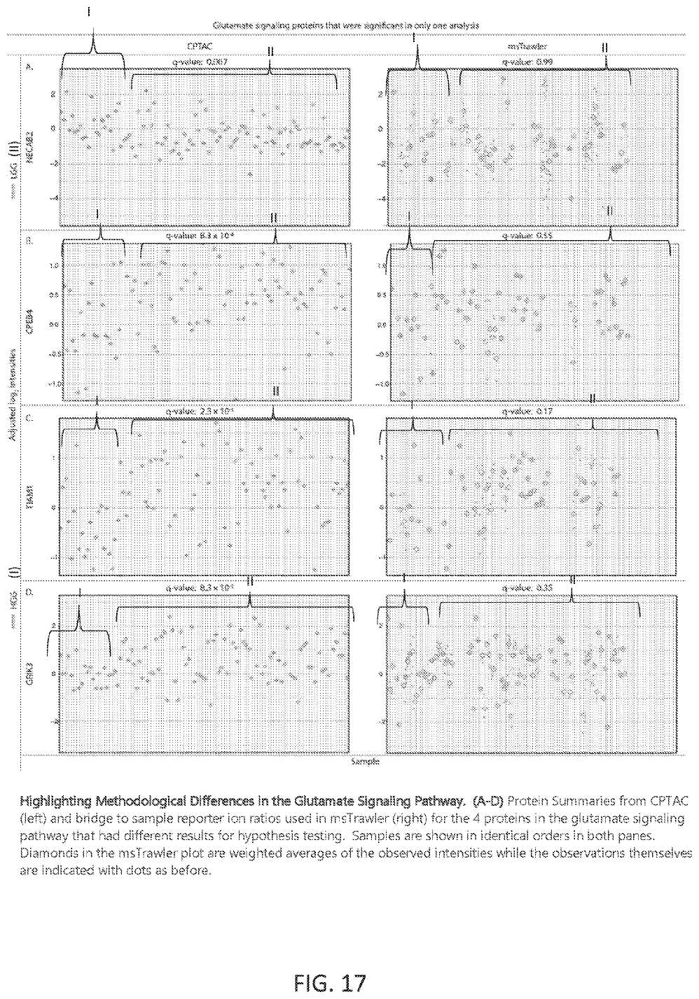

The pattern where imputations contribute to more significant findings was also found when focusing on one of the pathways featured in the original analysis, glutamate receptor signaling (GO:0007215). Glutamate signaling plays an important role in the transformation of malignant gliomas6,31 so it is potentially valuable that the CPTAC analysis reveals twice as many significantly altered proteins in this pathway (FIG. 16D). The two methods disagreed on the significance of four proteins: N-terminal EF-hand calcium-binding protein 2 (NECAB2), Cytoplasmic polyadenylation element-binding protein 4 (CPEB4), Rho guanine nucleotide exchange factor (TIAM1) and glutamate receptor ionotropic kainite 3 (GRIK3). None of these proteins were close to being significant in the msTrawler analysis (FIG. 17) and, as expected, many of these samples were missing in the msTrawler analysis resulting in smaller sample sizes and generally higher p-values (p-values are a function of sample size). Additionally, many of the observed proteins were represented by only a single peptide. In msTrawler the number of observations in each sample changes the confidence we have in a result, which played a substantial role in these methodological discrepancies. Unfortunately, access to the original tumors to perform further tests on these 4 proteins. However, RNA-Seq data was available and can be compared to the proteomics results.

Proteins found to be significantly different across histologies were split into groups based on the percentage of missing values and Pearson correlations between the RNA-Seq and proteomics results were computed within each subgroup (FIG. 16E). mRNA and protein changes show high and consistent correlations across methodologies in the subgroup with the fewest missing values. However, as the percentage of missing values increases, the methodologies diverge, with the CPTAC method showing a substantial drop in correlation. This pattern is observed in all 21 pairwise comparisons between cancer types suggesting that inferences dominated by imputations may not be as reliable as those made with a complete-case analysis.

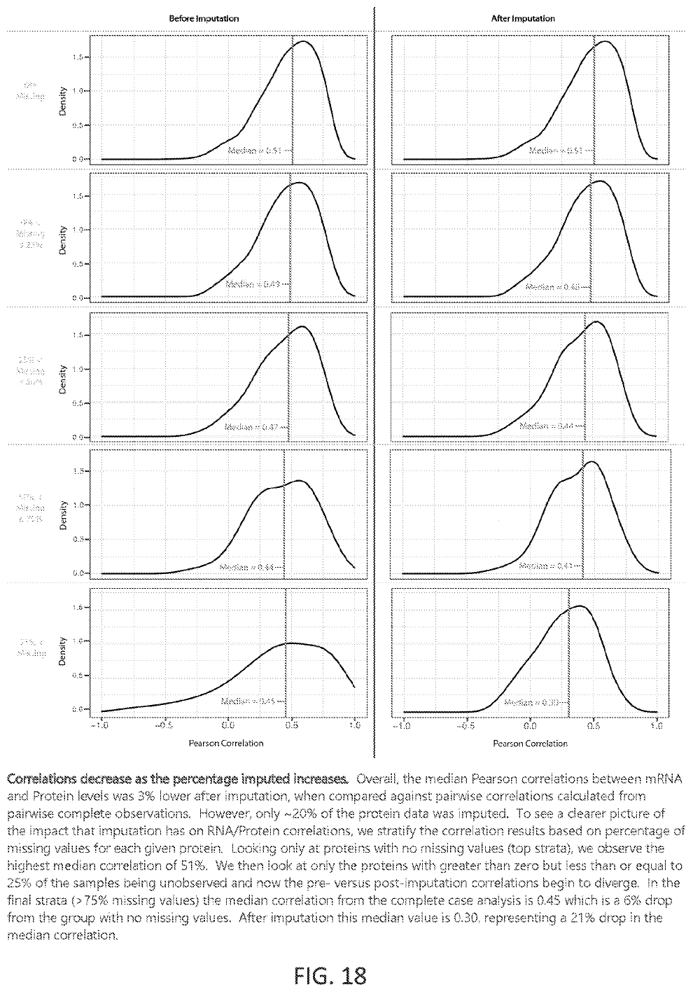

The correlations (FIG. 16) were taken across subsets of the proteome showing agreement between differential levels of mRNA and differential levels of protein abundance. A related correlation, dependent on a different set of biological and technical factors, can be calculated within each gene summing across samples. Comparing these correlations on the pre- to post-imputed data, it was observed that the median Pearson correlation drops by 0.03 after imputation. Stratifying the proteins according to the percentage of missing values reveals consistent decreases in the correlation between sets with the least to the most missing data (FIG. 18). This pattern holds when using both the imputed (0.51 to 0.30) and pre-imputed data (0.51 to 0.45), but in each stratum the correlation is higher when using the observed data alone.

Increasing the sample size with imputations likely increases the number of true discoveries in the experiment. However, the breakdown in mRNA/protein correlations suggests that the imputation procedure may not always work as intended and there is no way to separate successes from failures. Assumptions of missing data models are generally non-verifiable and considering the substantial difference in results that occurs due to missing data handling, many researchers may prefer to avoid imputations altogether. The msTrawler workflow makes it possible to avoid imputations while still using the full set of observations. Encouragingly, msTrawler was able to generate significant findings from the incomplete data even when the amount of missing data exceeded 75%, and the correlations with mRNA were consistent across of each strata of missing data (FIG. 16E).

### Example 8—Absolute and Relative Abundance

Reporter ion signals are typically presented as either intensities or signal-to-noise ratios (SNR). Respectively, these measurements are referred to as ion fluxes (intensities are adjusted for ion injection time) and ion counts (SNR is a surrogate for the number of ions collected in each scan1-3). To explore the relationship between these measurements and absolute abundance, two publicly available yeast datasets were compared. A commonly used TMT benchmarking dataset, the triple knock-out standard (TKO)4, provided MS3 reporter ion signals from Saccharomyces cerevisiae with minimal variability across co-isolated samples. We compared statistics from the TKO data to unified abundance estimates from the Saccharomyces Genome Database (SGD). The unified estimates incorporate data from multiple technologies (including MS1 based proteomics experiments) offering a unique resource for observing absolute abundance across an entire proteome.

Reverse hits, contaminants and the three proteins used in the knockout experiment were all removed from the TKO data. The data were further filtered by removing all peptides with summed signal-to-noise ratio (SSN) less than 200 or an isolation purity less than 0.75. TMT signals were summed across all channels to generate a single reporter ion statistic for each scan. The scan level statistics were either summed intensities, SNRs or de-fluxed intensities (intensities multiplied by ion injection time). The scan statistics were then further aggregated to create statistics for individual proteins (averages, sums and spectral counts, which are just the number of scans matched to the protein) and these, along with summed and average MS1 precursor intensities, were correlated against the unified absolute abundance data from the Saccharomyces Genome Database (FIG. 19).

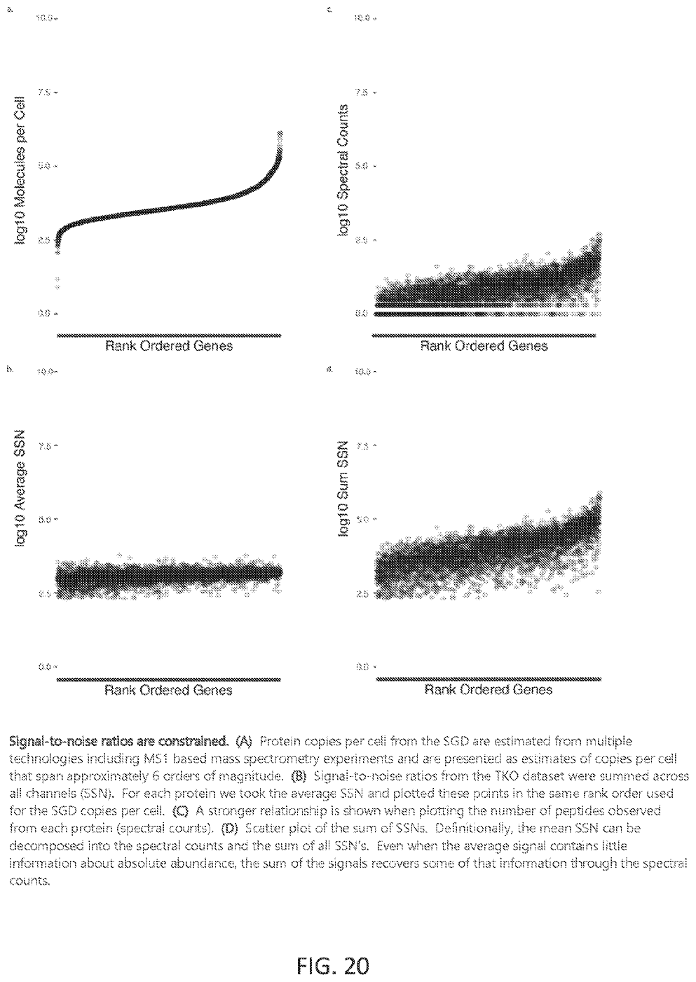

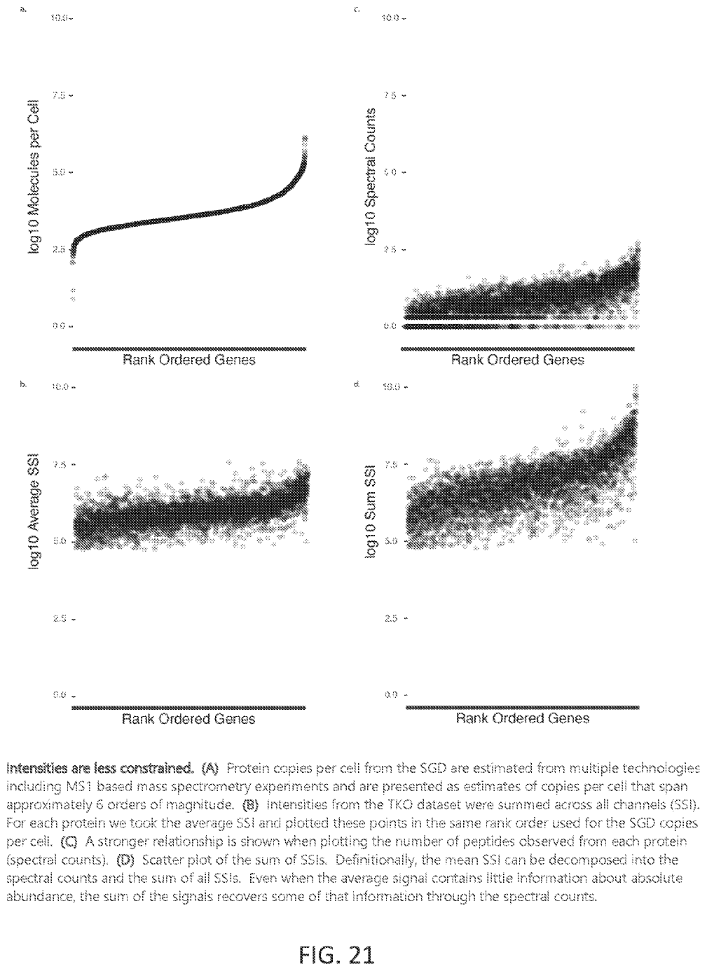

Scatterplots (FIGS. 20-21) show that the average reporter ion signal-to-noise ratio (SNR) has very little correlation with absolute abundance (0.35). The average intensity has a better correlation (0.61), likely because fluxes do not share the same upper bound that spatial constraints place on ion counts6 (FIG. 21). However, neither measure correlates as well with abundance as the average MS1 precursor intensity (0.73).

With all measures, the correlations are improved by taking into account the number of scans belonging to each protein (either through a sum or just the count). This is because the number of identified peaks from each protein is itself correlated with abundance. In general, the closer each statistic gets to measuring all of the ions generated from each protein, the better the correlation becomes. At the level of individual reporter ion scans, the correlations are weak and technical variation can dominate the absolute magnitude of each signal. While these statistics are still correlated with abundance, the true value of reporter ions come from the ability to precisely estimate relative, not absolute, abundance.

### Example 9—Heteroskedasticity

It is known that signals from isobaric proteomics experiments increase in variability as the “signal” magnitude decreases. The nature of this variation has previously been attributed to the process of sampling a small numbers of ions in each scan and while some efforts have been made to account for the heteroskedasticity either through modeling or transformations, the most common approach is to define a cutoff at which the observations are deemed too variable to be useful. Known ratios from the Interbatch Experiment (as described herein) provide a good opportunity to explore heteroskedasticity. Grouping all intra-batch ratios based on the magnitude of a fold-change, along with the dilution group of the denominator reveals that the precision is dependent on the signal magnitude. Also, for larger fold-changes, compression leads to both inaccuracy and increased variation (FIG. 9).

### Example 10—Signal-to-Noise Ratios (SNRs) as a Measure of Technical Variability

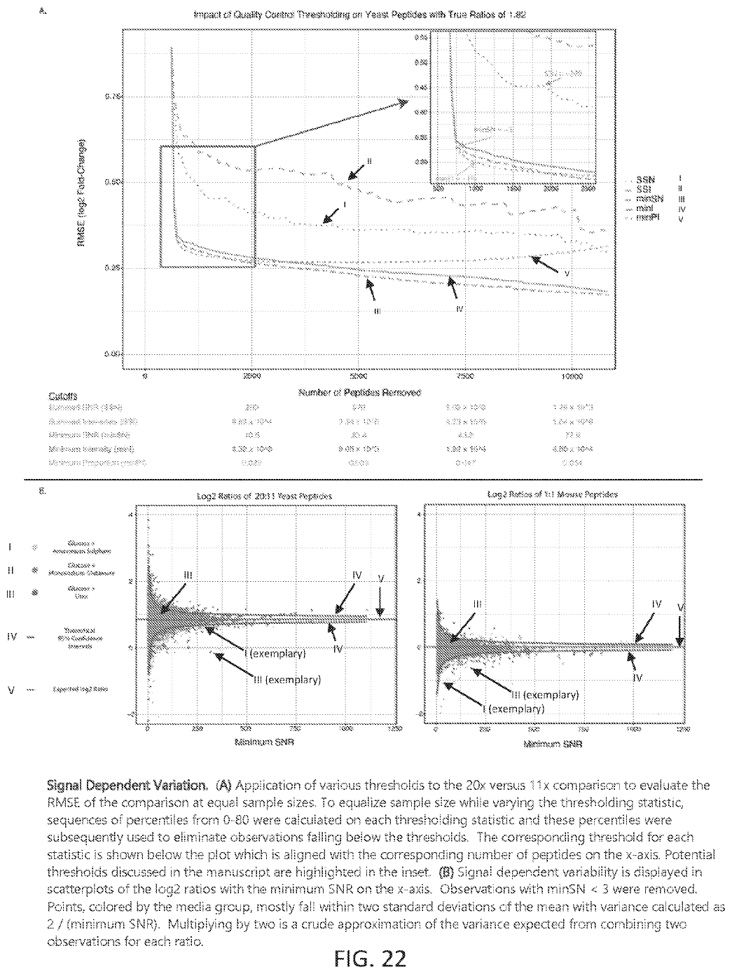

Removing highly variable measurements with a quality control (QC) threshold was explored, which began with a minimally filtered (SSN>20, which is just above what would be expected if every channel was at the noise threshold) dataset from The Interbatch Experiment and calculated root mean squared error (RMSE) after the application of various QC metrics. Continuing the focus on the 20× versus 11× comparison, percentiles of various potential alternative QC measures were calculated, including SSN, summed signal intensities (SSI), the minimum signal-to-noise ratio from each pair in the ratio (minSN), the minimum intensity (minI) and the proportion of the intensity (minPI=minI/SSI). Filtering the data at equal percentiles of each measure enabled to plot the RMSE of the ratios while removing an equal number of observations based on different thresholding criteria (FIG. 22a).

In this unbalanced dataset, it was observed that the measures of individual pairs of reporter ions (minI, minSN and minPI), successfully reduce the error with far less data loss than the aggregate measures (SSN and SSI). It was also observed that for both aggregate and individual metrics, signal-to-noise ratios do a better job of explaining variability than reporter ion intensities (note that the relative metric, minPI, is approximately the same whether we use intensities or signal-to-noise ratios).

Following the minSN curve from left to right, it was observed that no RMSE was reported until 617 observations were removed. These were ratios where the denominator had no reported signal and are not particularly interesting. However, the removal of the next 128 peptides resulted in a steep decrease to the RMSE of the remaining observations. Continuing past a minSN value of approximately 3, the decrease in RMSE follows an approximately linear relationship.

Limiting the dataset just to observations with SNRs greater than 3 reveals a very consistent relationship between variability and the minimum SNR in each pair (FIG. 22b). Curves representing two standard deviations of a Gaussian distribution with variance 2/(min [symbol] (SNR)), suggest that the inverse of the SNR approximates technical variability for both mouse and yeast peptides. In the theory of linear models, weighting observations by the inverse of their variance is known to minimize estimation error among all unbiased estimators. This suggests that using SNR's to weight each observation could be beneficial and msTrawler therefore makes full use of this relationship.

### Example 11—Interference and the Limit-of-Quantitation

The previously explored ratio of 1.82 (FIG. 22A) showed that a LOQ was likely to exist, but the number of observations available to study was fairly small. To observe the properties near the floor of the signal, the 1:18 ratio between channels 128c and 132c in batch 5 were highlighted instead. For this comparison, the co-isolated samples are often from larger dilution groups and the 18× fold-change should be sufficient to challenge our ability to accurately measure the denominator.

In a classical LOQ problem, if some observations are seen above the LOQ and others fall below, then setting the LOQ as a floor (any value less than LOQ is set equal to LOQ) can result in truncated ratios (FIG. 10). Since this is likely to result in conservative estimates of relative abundance, truncating SNRs at a value of 1 (LOQ equals the reported noise) has been implemented in practice. For the 18× versus 1× ratio, we anticipate that the set of ratios altered by any non-zero LOQ floor (the ratios where one of the observations was below the LOQ), should be, predominantly, less than 18. This expectation was reinforced by the standard model for TMT-interference, which in the context of this experiment, anticipates ratio compression whenever a mouse peptide was co-isolated with a target analyte (co-isolated yeast peptides would simply provide additional TMT ions matching the yeast dilution profile). Contrary to the expectations, it the systematic ratio expansion is observed instead (FIG. 11).

Setting the LOQ to an SNR of 1 revealed that not only did many of the values fall above 18, they were also far larger than would be expected by random error (FIG. 11a). Understanding the source of these large ratios (which we call ratio expansion) proved very difficult when relying only on sampling variation or TMT-interference. It was hypothesized that the benchmarking experiment had somehow generated a substantial number of non-zero interfering signals that were independent of the number of TMT molecules entering the Orbitrap from the target analyte. If true, then the observed ratio expansions might have been exacerbated for peptides that contain lysine, since on average the target signals would be larger than the interfering signals.

TMT is composed of an amine-reactive NHS ester group that reacts with the N-terminus and internal lysines of each peptide. Accordingly, for every peptide in the mass spectrometer that makes it to the point of fragmentation in the quantitative scan, we expect exactly one TMT molecule to enter the orbitrap for peptides with no internal lysine residues. For peptides with one internal lysine, we expect to see two TMT molecules. More specifically, since this experiment used synchronized precursor selection (SPS)3, it was expected that for peptides with an internal lysine, some fragmentation events result in the collection of two TMT labels while others only provided a single TMT label. If the denominator in the ratios was comprised of pure interference, the interfering signal would not necessarily share the mapping between peptide sequence and the associated number of TMT molecules. Consequently, it was predicted that ratios with an internal lysine would, on average, be larger than those without.

Raising the LOQ across three potential SNR thresholds (1, 5 and 10) show boxplots of the truncated ratios broken down by lysine content and the presence of a missed cleavage. (FIG. 11). Increasing the SNR threshold from 1 to 5 to 10 did eventually bring the 3rd quartile of ratios below 18. However, the differential results based on lysine content remained. Both the expected ratio compression and the lysine differential were eliminated more efficiently when using a relative rather than an absolute LOQ (FIG. 11-B). These relative LOQ values (defined as a percentage of the total signal) were calculated by finding the signal proportion that resulted in an equal number of truncated ratios to the corresponding absolute (SNR based) LOQ. It was expected that when a high enough number of peptides were collected, the systematic ratio expansion would be eliminated. However, it was noticed that both ratio expansion and the lysine differential were removed more efficiently when using a relative LOQ, suggesting that the nature of the problem was related to the magnitude of the other co-isolated analytes.

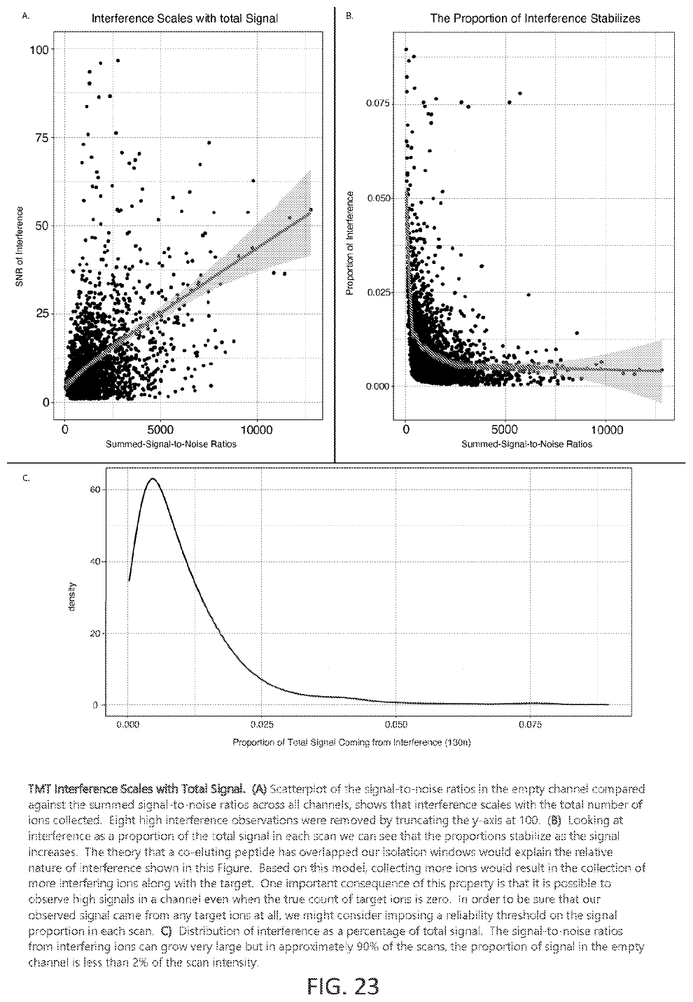

Interference is usually explained by the presence of isobaric tags on co-isolated, non-target peptides. When this happens, collecting more ions simply results in the collection of more interfering ions. Consequently, the interfering signal scales with the total signal in the scan. Interference was observed directly by plotting the empty channel from batch-4 (FIG. 23). There were no yeast peptides in this channel, so all the observed signals come from either missed identifications or interference. It was noted that the general trend of the SNRs from the empty channel increased with the total signal (FIG. 23-A), while the proportion of signal coming from the empty channel stabilized as SSN increased (FIG. 23-B). This relationship between interference and total signal should be expected under the standard model of TMT interference, but it is also consistent with other potential sources of interference, such as artifacts from the Fourier transform used to generate the reported signals, or isotopic contamination, both of which would generate interference that scales with the total number of TMT ions collected from co-isolated samples.

### Example 12—Ratio Expansion and Isotopic Impurities

Based on the Interbatch Experiment (as described herein), it was anticipated the existence of “noise” signals that met the following conditions: 1) The noise does not match the expected patterns from any peptides in the dilution experiment (ratio expansion was observed rather than the usual compression from TMT-Interference); 2) The noise is not proportional to the signal in our numerator (if it were, the observed lysine differential would not exist); 3) The noise is dependent on the signals from co-isolated samples (suggested by the improved detection when using a relative LOQ).

To further explore the ratio expansion phenomenon, a Ratio Expansion Experiment was designed that placed a small amount of yeast protein in a denominator while varying the magnitude of the immediately adjacent peaks. This design created ratios from denominators with a potential source of interference immediately on the left (TMT tags 129C-132N), with no flanking peaks (132C-135N), and immediately on the right (132C-135N, FIG. 13a).

Additionally, many of the experimental configurations were altered from the Interbatch Experiment to exclude these factors as potential causes of the ratio expansion. To this end, the following was changed: isobaric label (TMT 16plex to TMTpro 18plex), interference model (mouse plasma to a PC9 human cell lysate) and pre-processing software (MassPike to Proteome Discoverer from Thermo). Additionally, the samples were ran in triplicate using both the original SPS mass window as well as with a more restricted mass window to exclude y1 ions (lysine containing peptides which would be more likely to share y1 ions with interfering peptides). Finally, pre-processing of the data was performed both with and without corrections for isotopic impurities in the TMT reagents. Ratio expansion persisted in this experiment across all these modifications except for the variation where TMT isotopic impurity corrections were not included.

To visualize ratio expansion, plots of the upper decile of the observed log 2 ratios minus the true log 2 ratios determined by the dilutions were generated. The upper deciles of these deviations were calculated conditional on the observation meeting our LOQ criteria.

Absent the implementation of a LOQ, only intensities of zero were removed, which represented 1.9% of both the raw intensities and isotope adjusted intensities (adjusted for isotopic impurities; FIG. 13b).

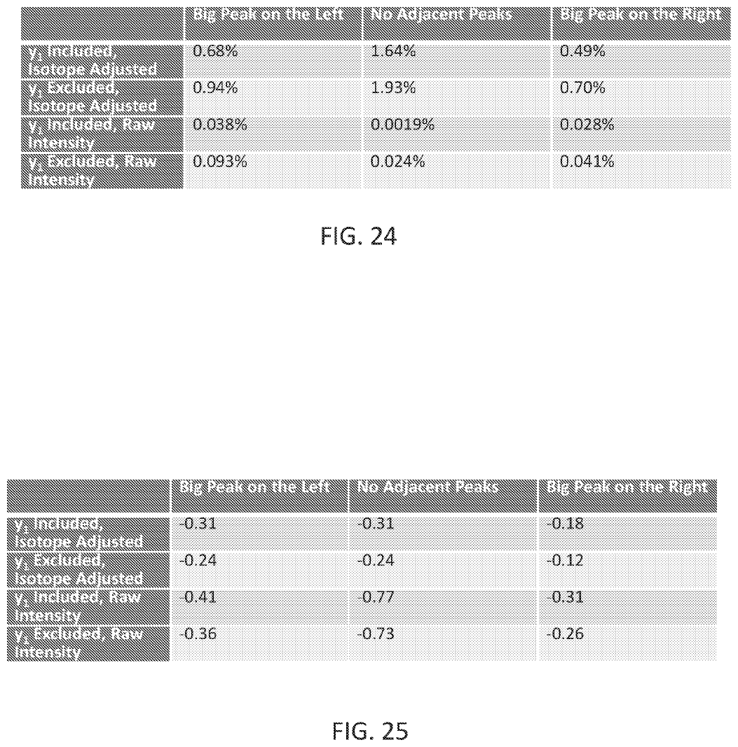

The first important observation from The Ratio Expansion Experiment is that ratio expansion was still present in this additional dataset and does appear to be highly dependent on the configuration of the samples. If ratio expansion is defined as any ratio more than twice as large as expected, then 1.64-1.93% of the ratios in in the 129C-132N subgroup were expanded, which is more than twice the occurrence observed in either of the other groups (FIG. 24). Notably, the absence of a large adjacent peak next to the denominator in this group suggests that the isotopic purity modification was likely to be purely subtractive. The second and most important observation in The Ratio Expansion Experiment is that when using raw signals, with no adjustment for TMT isotopic impurities, the phenomenon of ratio expansion essentially disappears (FIG. 13b).

Exclusion of the y1 peak from SPS selection did not have a substantial impact on ratio expansion. However, consistent with the theory that excluding the y1 reduces TMT-interference, it was observed less ratio compression in the median ratios of every subgroup with the reduced SPS mass range (FIG. 25). While this is an interesting finding, the most substantial observation in the experiment is that using raw signals, with no adjustment for isotopic impurities, the phenomenon of ratio expansion essentially disappears (FIG. 13b). Adjusting for isotopic impurities is done on a scan-by-scan basis. The process requires estimating the corrections that should be applied to each scan based on the reported percentage of isotopic impurities from the vendor (Thermo). As with any estimation problem, the estimate is imperfect and has an associated error. In both the Interbatch and Ratio Expansion Experiments the errors from these adjustments are likely to be comparable in magnitude to the observed signals from the less abundant channels.

Consequently, situation where the error from the isotopic adjustment results in small denominators and surprisingly large ratios is being encountered. Finally, it should be noted that estimation error from this process meets all the criteria for the “noise” described in the beginning of this section.

While the lack of ratio expansion suggests a benefit to skipping the isotopic adjustment entirely, it is important to note that this would not be recommended as it was also observed that the median ratios were improved, in every subgroup, by the same adjustments that seem to cause the expansion FIG. 25). Ratio expansion will likely generate hundreds of potential outliers and false positives, which are very notable in experiments where most analytes do not change abundance. However, that total represents <2% of the data and compromising the quality of the remaining 98% of observations seems ill-advised. Furthermore, if the isotopic adjustment gives reason to believe that most, or all, of a given signal came from impurities, it would be very strange to ignore that information. Fortunately, implementing a limit-of-quantitation allowed to identify these cases without compromising quality in higher signal measurements.

The upper deciles observed after applying various LOQs show that ratio expansion, in the remaining observations, decreases as the LOQ is raised (FIG. 13c). Consistent with results from the Interbatch Experiment, it is observed that using a relative LOQ removes the expanded ratios more efficiently than an absolute measure. However, identifying observations that fall below the LOQ is only a partial solution, as it what to do with these observations need to be determined.

Although observations below the LOQ, including intensities with zero signal, were not measured with the same precision as other data points, they still contain a large amount of information. For example, it is likely that the true values, had they been observed, would have been lower than the LOQ. To see why removing these cases could be undesirable, consider a large study from a human population with a rare genetic disorder that causes a substantial drop in the levels of a specific protein. In this situation, eliminating observations below the LOQ could result in missing the effects of the disorder entirely.

Imputing a LOQ can preserve these relationships and it has been shown that imputing the LOQ/2 tends to work well across many possible simulated scenarios. In reality, it is uncertain where the actual observation would have been if the technology did not have a LOQ, so treating these imputations the same as real observations has numerous potential drawbacks. Fortunately, msTrawler provides a framework for modeling intensities with weights defined by SNRs. It is trivial to simultaneously impute both an intensity value and a SNR so that the imputed observations are always given less consideration than the real observations that were above the LOQ.

For the remaining analyses described herein, the LOQ is defined as 1% of the summed intensities in each scan. For each observation with a SNR less than the LOQ, both a new intensity and a new SNR is simulated to be used as a weight in our models. To avoid generating observations with no variance at all, intensities were randomly imputed with a small amount of variability: A Gaussian random variable with an expected value of log ¬¬2((SSI*0.01)/2) and variance of 2/(SSN*0.01) is generated and then exponentiated. The log/exponential step ensures positive intensities. SNRs that were later used to assign weights in our models were also imputed at (SSN*0.01)/2. With this strategy, it was aimed to eliminate error caused by ratio expansion, and preserve the ability to detect changes that exceed the dynamic range of our experiments, while mitigating error introduced by relying on an imputation.

The realization that isobaric proteomics experiments have a non-zero LOQ, has implications for all studies with high variability across samples. However, a relative LOQ takes on critical importance for specialized experiments that, by design, generate a large dynamic range between isobaric tags. This is common in isobaric experiments for estimating the stoichiometry of post-translational modifications, and some methodologies for single-cell-proteomics. The properties of low-signal intensities described in this manuscript are very likely to play a role in the quantitative challenges previously described for single-cell proteomics.

### Example 13—Experimental Design and Strategies for Removing Interbatch Variability

The composition of co-isolated samples in conjunction with temporal variations in analyte flux results in substantial variation across scans. The removal of this technical variation is of the utmost importance for performing successful data analyses. The literature is dominated by efforts to achieve this reduction in variability through “normalization” to either a measure of central tendency (mean or median intensity) or to a signal from a standard sample (bridge channel). Statistical methods for random block designs provide an alternative to normalization with the potential to prevent a few failure modes of the normalizations. However, none of these options are satisfactory when designing tools to handle arbitrary experimental designs.

Row Normalization: In this section, “normalization” will be referred to as the process of dividing each intensity in a scan (or subtracting with log intensities) by some “row normalization factor”. The row normalization factor will typically be either the average (mean or median) of the scan, or it will be the observed measure from a bridge standard. Arguments have been presented against the use of bridge normalization on the grounds that variation in this signal will likely be greater than the variation in the average, and this error will be propagated into all signals in the scan. This concern is especially poignant in cases where the bridge sample itself has very low intensities and may be less reliable than the rest of the observations in the scan. However, while it is certainly true that taking averages will reduce sampling error, the argument is heavily dependent on experimental design.

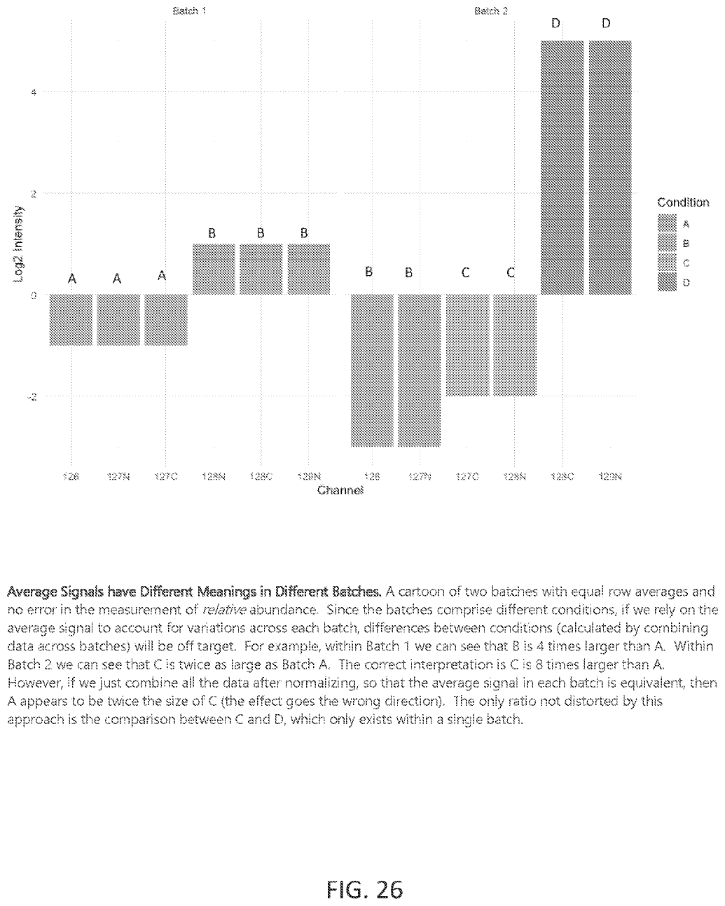

The normalization procedures can be thought of as a transformation that alters the interpretation of every datapoint. From this perspective, the transformed values represent deviations from the average sample and if the interpretation of the average sample changes across batches, the normalization strategy may produce poor results (FIG. 26).

In statistical nomenclature, the design in FIG. 26 is an example of an unbalanced, incomplete block design. Balanced and complete block designs are two important concepts in the field of experimental design. The term block refers to a grouping of samples that share some common characteristic, distinct from the subjects of inquiry, that may influence the response variable. In other words, a block is a set of samples that share a nuisance variable. In other fields, common blocking variables are farm plots or hospitals. In the context of isobaric proteomics, every scan creates a statistical block. These scans are nested within batches (groups of co-isolated samples), and it is at the level of each batch where the decisions about how samples will be distributed can be informed by the concepts of experimental design. A complete block design is one in which all treatments are tested within each block, while a balanced design is one in which all treatments or experimental conditions are applied an equal number of times in each block. Balanced, complete block designs utilize randomization to minimize the impact of a nuisance variable and data analysis strategies based on these designs will often minimize estimation error.

Multiple studies on proteomics methodology have reported poor performance when using average row normalization in incomplete and imbalanced designs, while the manuscript arguing in favor of this approach only did so in the context of a complete, balanced design. In such a context, the average row normalization should work well, and msTrawler will subtract the average log signal from each scan whenever a bridge channel has not been specified. However, for general purpose designs the average row normalization cannot be recommended.

The reference error, described in FIG. 26, will depend on the experimental design and it only applies to normalization factors based on average intensity (bridge normalization should still work). However, there is another problem with row normalization that applies to both strategies. Isobaric proteomics data is heteroskedastic. One of the major findings of this work is that combining multiple batches of proteomics data requires controlling for the number and quality of observations in each batch. This information is lost when performing row normalizations. To see this, consider the strategy of simply summing up all the peptide intensities in each sample. As the number of observations increases, so does the sum. As the magnitude of the signals increase, so does the sum. But the same thing happens to the signals from the bridge, and the average signals in the batch. So once the data is normalized to the row normalization factor, the information about number and quantity has been removed from the statistic.

One last flaw that applies to both bridge and average row normalizations, is that they remove the blocking structure from the data. While it can be hoped that the normalization accounts for the most important influence of a block design, more subtle impacts may persist. In particular, there will be no way to place more weight on comparisons that are being made within versus between blocks. Differential weighting of contributions to an estimate based on blocking structure is a standard part of traditional statistical modeling for block designs that cannot be implemented if the blocking structure is discarded after applying row normalizations.

Categorical Random and Fixed Effects: The traditional approach for analyzing data from experiments with random block designs is to incorporate the nuisance variable into the statistical model, either by estimating an additive effect for each level of the blocking variable (fixed effect for block) or by imposing a correlation structure between observations that share a nuisance parameter (random effect for block). Both strategies, when applied correctly, will avoid the type of reference error highlighted in FIG. 26. In the presence of a fixed effect for each block, ordinary least squares estimates will consist of contrasts taken within each block, or by connecting the within block contrasts through shared treatments (difference of differences).

The problem with using fixed effects is that observations that might otherwise be used to estimate the parameters of interest are now required to estimate nuisance parameters. In complete designs with multiple samples per treatment group, this will simply result in a loss of power. However, in the general block design, the lost degrees of freedom can result in situations where the block and a particular parameter of interest will be confounded. If a sample must be used to estimate a fixed effect, it is preferable to accomplish this with a bridge sample. However, another method for avoiding this loss is to instead utilize a random effect for each block.

In a linear mixed model, random effects do not alter the expected value of the observations (the random effects are constrained to have a mean of zero). Consequently, the averages are entirely determined by the fixed effect structure while the random effects alter the covariance structure. For an intuitive understanding of how these models work, one can imagine first estimating treatment effects without any consideration at all for the blocking variable, and then subsequently partitioning the variability into the within- and between-block variance components. The between block variance is then removed as part of the procedure for statistical inference. Unfortunately, the success of this approach is entirely dependent on the ability to accurately estimate variance parameters, which requires a sufficiently large number of levels of the blocking variable.

The number of blocks necessary to reliably use random effects is a matter of debate. In the case of a design with balanced incomplete blocks, Henry Scheffe\acute{ } considered having more blocks than treatment effects to be a prerequisite for the method. Specific guidelines have been developed, but even the most permissive of these rules, if applicable, would result in widespread failures for proteomics data. For less abundant proteins, it should be expected that only a few scans will be observed in each batch (and their detection will be inconsistent across batches). For these proteins, models that depend on random effects to account for scan-to-scan variation are liable to produce very undesirable results. In cases where a few observations show large scan-to-scan variation, the between block variation may be overestimated and the residual error underestimated—resulting in absurdly small p-values. Researchers could utilize this method and simply discard the less abundant proteins, resulting in a costly process.

The msTrawler Bridge Model: Statistical models for block designs do not traditionally provide a means for handling bridge samples. This is because the ability to incorporate a bridge standard into every block is a rare and powerful technological advantage of isobaric proteomics. For the same circumstance to arise in the context of a multicenter clinical trial, the ability to treat the exact same patient in multiple centers would be needed. Typically, this is not even theoretically possible as treatments usually can't be administered more than once, to say nothing of the practicality of sending patients all over the world to receive redundant treatments.

For many reasons, a bridge standard should not be thought of as simply adding an additional treatment group to each block. The first and most obvious reason, is that incorporating a bridge channel does not actually add any treatments to an experiment (the new sample is strictly incorporated to remove technical sources of error). But there are other crucial differences between the bridge and other multiplexed samples. In arbitrary designs, samples will be associated with many covariates, e.g. smoking status, sex, age, etc. The bridge sample does not have a smoking status. Finally, the bridge does not share the same sources of variation as typical samples distributed throughout the blocks. A regular patient sample will be randomly assigned a treatment, so that differences in individual responses to a treatment are likely to be evenly distributed across groups. While randomizing across groups should equalize inter-individual variation across groups, that inter-individual variation will still be present in the data, but it will not exist for the bridge sample. The bridge is not a random selection from a population, it is actually the identical sample being analyzed in each block.

While the bridge sample does not fit nicely into the standard models for block designs, these can still be incorporated into arbitrary statistical models. The key is to realize that the purpose of the bridge is account for scan-to-scan variability and that the proper interpretation of the non-bridge samples is as a deviation from the bridge. To this end, described herein is an algorithm (for use with any system or method described herein) to modify any non-degenerate design matrix to incorporate fixed effect nuisance parameters for each scan. First, a design matrix is created for a given experiment while ignoring the existence of the blocking variable. This will be referred to as the Base Model.

With the Base Model established, a strategy is then developed for incorporating bridge channel intensities to account for scan-to-scan variability. Let S denote the number of scans present in the data and let X denote the design matrix generated by the Base Model. Then the design matrix can be expanded by adding S rows and S columns. Each new column defines a scan and each new row corresponds to a bridge channel intensity. All entries for columns from the original design matrix are set to zero.

This approach generates a design matrix that will be full-rank for any X that was full-rank prior to considering blocking on scan (see Example 14 for an example). As such, the approach is fully generalizable for arbitrary experimental designs, and preserves both the blocking structure and information about signal quality found in the untransformed data.

Accordingly, row average normalization does not generalize across unbalanced designs. However, for complete, balanced designs, it is expected that a row average normalization will work well. In the absence of a bridge channel (e.g. just a single batch) msTrawler uses this strategy.

Bridge normalization does work across arbitrary experimental designs. However, it results in a loss of information regarding signal quality and blocking structure. When a bridge channel is present, it is preferred to incorporate the bridge directly into our statistical modeling.

Random block effects perform poorly when the number of blocks is small. Given the current dynamic range of proteomics experiments, and the widespread missingness of low abundant analytes, even very large experiments are likely to contain a substantial number of proteins with a small number of scans.

Incorporating the bridge scans to estimate fixed effects for each nuisance parameter is a very flexible solution. It works with arbitrary designs and enables modeling that accounts for signal quality in both the bridge and target samples, while preserving batch structure. Since batch structure does matter in these models, some thought should be given as to how samples are distributed. Random assignment of samples should be optimal, however if some comparisons are of more interest than others, it would be advisable to place the most important comparisons in the same batches. As an example, if longitudinal drug treatment in multiple patients is being studied, the effect of the drug is most interested and not comparisons between individuals, it is expected that the best results would occur when each patient time course is placed within the same batch. This results in a loss of accuracy when estimating differences between patients, but minimal variance in the individual time trends.

### Example 14—Algorithms and Statistical Models

Column normalization: Statistical modeling will be performed independently for each protein. However, the existence of systematic effects impacting all the measurements in each sample due to experimental factors is not anticipated, such as pipetting errors or digestion variability. Typically, these systematic effects are removed by equalizing the summed reporter ion signals within each channel (columns of a matrix with scans in each row and samples in each column). The strategy is motivated by the assumption that the total amount of protein is unchanged across samples, which implies that deviations are purely representative of technical artifacts.

The assumption of equal total protein content is often, though not always, forced to be true through experimental procedures that equalize total peptide content across each sample. Accordingly, for the typical experiment it would be perfectly sensible to treat deviations in the total abundance as technical artifacts. Unfortunately, for two major reasons the summed signals in each channel should not be thought of as a measure of total peptide abundance. First, reporter ions intensities, as we have discussed extensively, are not measures of absolute abundance. Second, we only observe a subset of the total proteome. Consequently, it is possible to observe a subset of reporter ion intensities such that the small number of proteins that do change, represent an outsized proportion of the observations. A technical artifact found in The Interbatch Experiment (as described herein) highlights the problem.

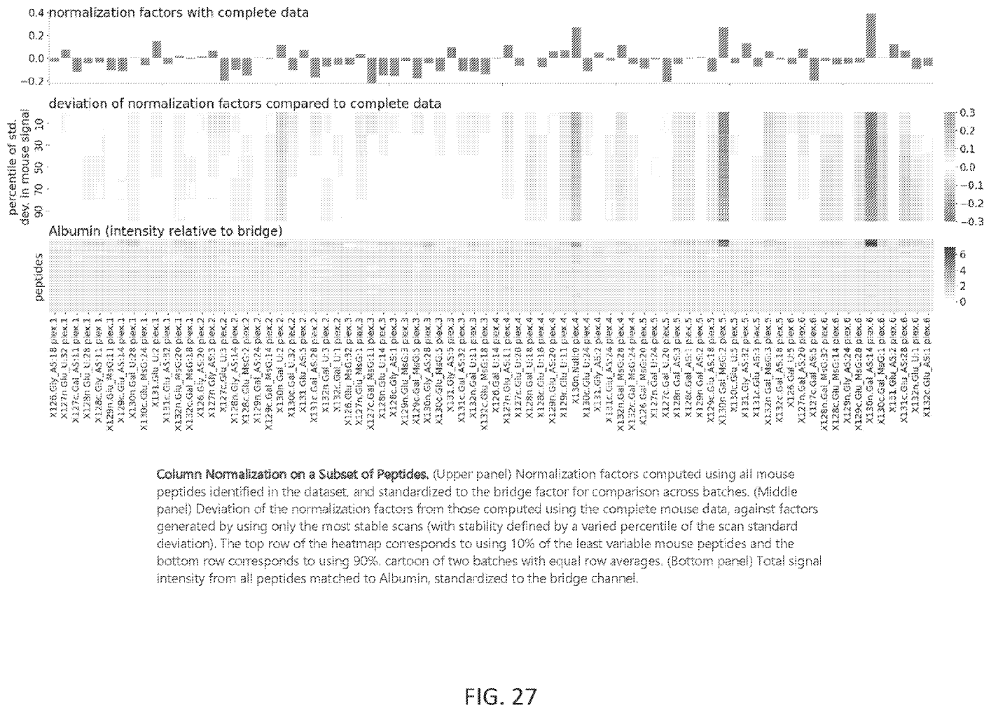

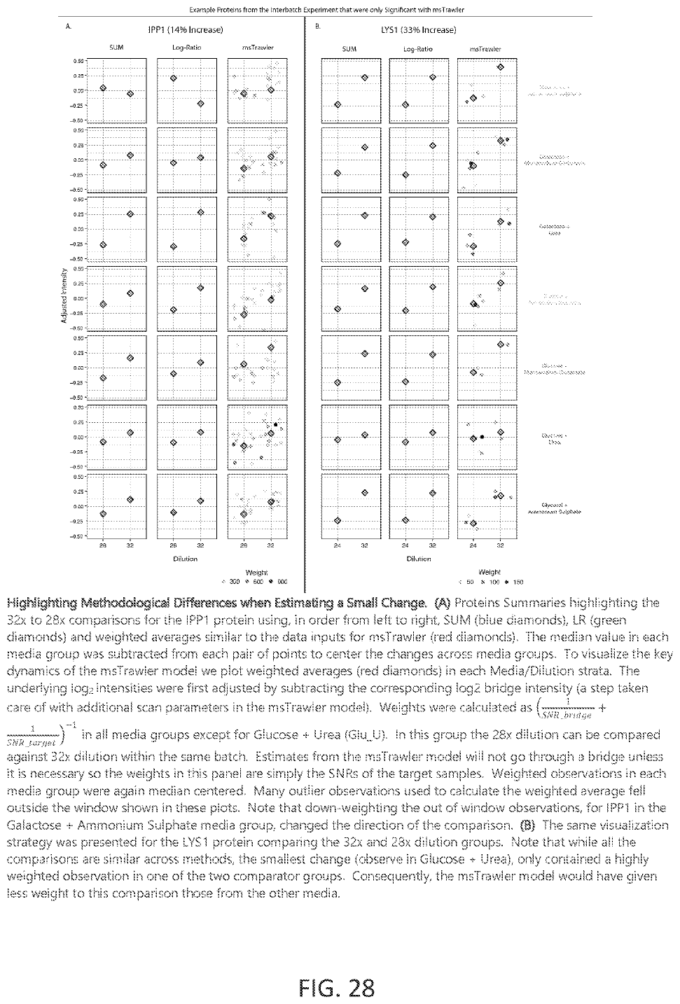

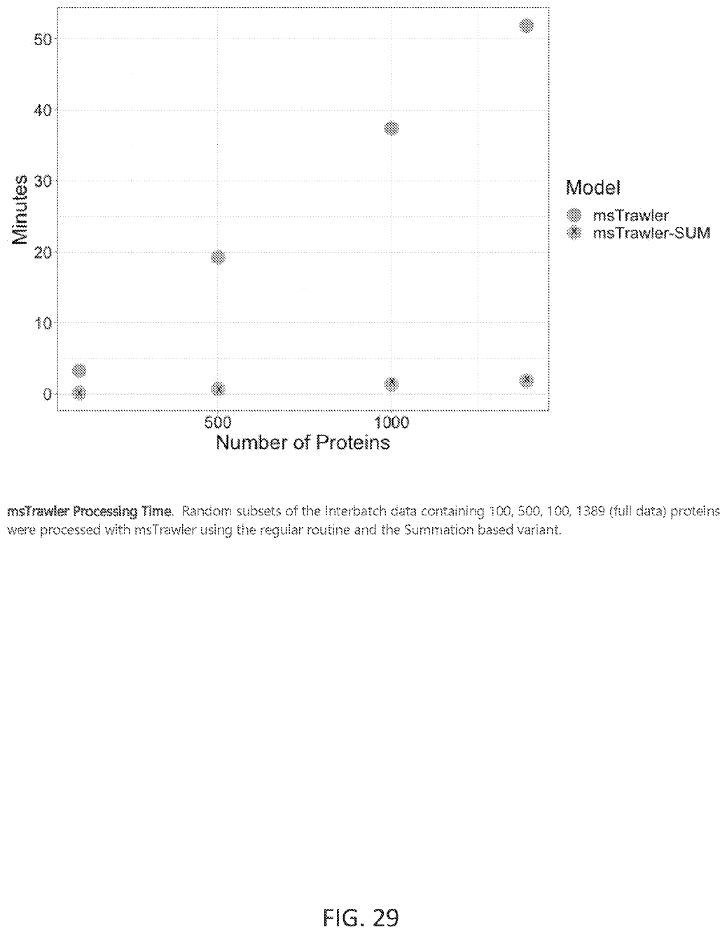

In The Interbatch Experiment, we performed column normalization by calculating the average log 2 intensity from the mouse peptides in each sample and then centering these sample averages at zero. The centered values, that we refer to as normalization factors (FIG. 27; top panel), are subsequently subtracted from all the observations (including yeast peptides), which are then re-exponentiated to ensure no negative values were induced in the process. In theory, normalizing the data in this way should remove the systematic effects from each sample. However, while studying the Interbatch data, it was noticed that an artifact in the experiment resulted in a small subset of mouse plasma peptides deviating from the typical trends.

In channel 130n (a specific TMT reporter tag), reporter ion intensities for a small percentage of peptides increased in magnitude with the batch numbers. One possible explanation is that this was the result of specific peptides (e.g. highly hydrophobic peptides) being preferentially retained during pipetting. Another possible explanation is that some type of contamination may have interfered with labeling efficiency for only a subset of peptides. In any case, a small percentage (<5%) of mouse peptides were observed to have high intensities in just a few of the channels. While few, some of these peptides came from highly abundant proteins.

Protein abundance in plasma is known to have a range of nearly 12 orders of magnitude. Given this large range, even small amounts of variation in any of the highly abundant proteins (e.g., Albumin, Serpin3ka, etc.) across samples can drive variation in the average (or summed) signals in each column. In these circumstances, more robust measures of the systematic trend such as the column median could prove useful.

The 130n artifact did impact several peptides from highly abundant proteins, including Albumin (FIG. 27; bottom panel) and it was observed that the yeast ratios corresponding to these channels were all a bit off target. To avoid including peptides with changing abundance in the calculation that first calculated the standard deviation of every scan in every batch. Then limited the scans used to generate normalization to only the “most stable” scans. Specifically, the bottom X % of mouse peptides were selected ordered by the standard deviation in their signal intensity and computed the normalization factors for X=[10, 30, 50, 70, 90]. It was observed that removing highly variable mouse peptides changed the normalization factors substantially, in a batch and channel dependent manner (e.g., channel 130n in batches 2, 4, 5, and 6) while they largely remained independent of X for X<90 (FIG. S11; middle panel). Most samples were unaffected by limiting the data used for normalization, while the factors corresponding to the 130n artifact were all reduced, resulting in deviations to the systematic shifts of ˜10-20%.

Based on this exploration, it was opted to use the 50% most stable mouse peptides for normalization in The Interbatch Experiment, and the 50% most stable of all peptides in the remainder of analyses. Similar results can be achieved by taking a column median, but the sub setting strategy provides greater flexibility (for example in a protein turnover experiment one might assume that only 5% of the peptides will be stable).

Finally, while The Interbatch Experiment differs in many ways from a standard proteomics experiment, the existence of peptides that deviate from systematic sample effects should always be expected. The assumption that total protein content should be the same across samples never implied that all proteins should be the same across channels. If the ones that are changing represent a sizable proportion of the observed data, then using all scans to generate normalization factors may result in undesirable deviations.

Defining the Statistical Model for The Interbatch Experiment: To ultimately establish a statistical model that accounts for scan-to-scan variability, the statistical model that we would use if we did not consider scan-to-scan variability is first defined. This is referred to as the Base Model. If N reporter ion intensities were observed, let each log 2 intensity be denoted as yᵢⱼₖ where i=1, . . . , 11 indexes the categorical variables for each dilution group (γᵢ), j=1, . . . , 7 indexes the categorical variables for each media group (δⱼ), and k=1, . . . , K_{i,j} the number of scans observed in sample i,j. Then the Base Model is

$${y}_{ijk} = {β}_{0} + {γ}_{i} + {δ}_{j} + {b}_{ij} + {ϵ}_{ijk}$$

where the intercept, β₀, represents the expected value of an observation from media group 1 (glycerol+ammonium sulphate) and dilution group 1 (32×), i.e. γ₁=δ₁=0. The remaining fixed effects represent the expected difference between the reference group and the corresponding level of the parameter. Hypothesis tests of the form H₀: δⱼ=0 (differential abundance relative to the largest dilution group) will be the hypotheses of interest in the benchmarking experiment.

$${ϵ}_{ijk} \sim N ( 0 , \frac{{σ}^{2}}{{SNR}_{ijk}} )$$

is the residual error with a variance term that is inversely proportional to the SNR of the observation. Independent of ∈ᵢⱼₖ, bᵢⱼ˜N(0,τ²) is a random intercept for each sample. The inclusion of this random effect establishes the multi-level modeling framework in that we observe 7*11=77 physical samples, but N reporter ions within the various samples. bᵢⱼ establishes correlations between observations from the same samples that are essential for controlling type-1 error rates when modeling data directly from reporter ions.

Unfortunately, multilevel models are well known to have inflated type-1 error rates when sample sizes are small or when the observations within each sample are unbalanced. This is precisely the situation that most commonly arises in proteomics datasets. Fortunately, the Kenward-Roger adjustment appears to resolve the issue as it has across many other disciplines.

As described in Example 13, the design matrix from this model is converted, without regard to any bridge samples, to a modified version that incorporates the bridge intensities. Tbridge model for the benchmarking experiment becomes a model on the vector (yₛ, yᵢⱼₖ), where

$${y}_{s} = {α}_{s} + {ϵ}_{s} , if {y}_{s} represents a bridge channel ,$$

$${y}_{ijk} = {β}_{0} + {α}_{s} + {γ}_{i} + {δ}_{j} + {b}_{ij} + {ϵ}_{ijk} , otherwise ,$$

where s=1, . . . , S indexes the bridge intensities, αₛ is a nuisance parameter that accounts for scan-to-scan variability, and each combination of ijk indices maps to exactly one of S scans in the data. The error vector, (∈ₛ, ∈ᵢⱼₖ), represents Gaussian error with the same weighted variance structure described above.

This approach generates a design matrix that will be full-rank for any X that was full-rank prior to considering blocking on scan. As such, the approach is fully generalizable for arbitrary experimental designs, which enabled the flexible modeling strategy found in the msTrawler package.

Permitted Designs and Limitations: msTrawler generates models, like the one described above, based on a parameter file that specifies the experimental design. However, describing all possible experimental designs in a single parameter file is likely not a tractable problem. A solution was to allow for a large class of possible options.

A sample file associates metadata to individual samples and a covariate file provides more details about the sample information. The combination of these files allows for the inclusion of an arbitrary number of categorical and continuous covariates, ID variables that indicate the use of a random intercept model for repeated measures, and time variables that allow for linear, quadratic and cubic polynomial time trends.

Missing data patterns can often cause parameters in these models to no longer be estimable (extrinsic aliasing). To avoid the estimation of inestimable parameters, without requiring complete data, all possible model references are looped through, and each parameter is tested for estimability, only reporting estimates for parameters that pass the test (FIG. 8).

In addition to problems with estimability, sparse data may result in situations where a multi-level model does not make sense. The simplest case where this would occur is when only a single peptide is observed. When this happens, there are insufficient observations to estimate within-sample variation. To avoid attempting to fit models with insufficient data, the following requirements were imposed, for an experiment with p parameters, N samples, n scans and B batches.
1) There must be at least 3 more scans than batches to estimate σ: (n>B+3).
2) There must be at least 3 more samples than parameters to estimate τ: (N>p+3).
   If the above rules are not met, or if the model fails to converge for unforeseen reasons, the random effects are removed and fit the specified weighted regression using a standard linear model. In the applications described below, we only used results from the full msTrawler model. Note that p may vary across proteins due to missing data and these rules are applied after seeing which levels of each factor were observed.

### Example 15—msTrawler-SUM

msTrawler was designed to fit statistical models to multi-batch data while still accounting for the number and quality of the observations, which can vary substantially across batches. Within a single sample, it has long been recognized that the simple SUM implicitly accounts for variations signal quality since higher signal measurements contribute more to the summation. A similar logic applies to the number of measurements. As the number and quality of observations increase, so does the SUM. Consequently, it was hypothesized that an aggregated version of msTrawler, that kept track of aggregated SNR's and used them within the framework of the msTrawler bridge model, might perform comparably the mixed model while substantially reducing computational burden.

To this end, incorporated a parameter was incorporated into the msTrawler software called N_SUM. Whenever the number scans, post-outlier filtering, are less than N_SUM, the adjusted intensities along with the corresponding SNR values for each sample are aggregated. Setting this parameter to some very high value, e.g. N_SUM=99999, guarantees that models will always be fit with single number aggregates for each protein. Consequently, the mixed models will never be fit. Crucially, these numbers are also calculated for bridge channels, which in conjunction with the msTrawler bridge model means that we can account for batch composition without “normalizing” away the information about the number and quality of peptides contained in each summation.

Hayflick Model: For each protein, the published summaries from each sample were used as outcomes in the model

$${y}_{ltk} = {β}_{0} + {β}_{hTERT} + ( {α}_{1} + {γ}_{1 , hTERT} ) * t + ( {α}_{2} + {γ}_{2 , hTERT} ) * {t}^{2} + {ϵ}_{ljk} ,$$

where l=1 for observations from the RS line and 2 for observations from the hTERT line. Accordingly, when l=1, β_{hTERT}=γ_{1,hTERT}=γ_{2,hTERT}=0. t=18, 25, 29, 33, 37, 46, 50 are the PDL doubling times and k=1,2,3 indexes the replicates in the experiment. ∈ᵢⱼₖ is the standard Gaussian error term. This model was also used as the Base Model in msTrawler and in both models the primary hypothesis tests of interest are the test for an overall time trend in the RS line, H₀: α₁=α₂=0 and a test for a difference in quadratic time trends between the two cell lines, H₀: γ_{1,hTERT}=γ_{2,hTERT}=0

### CPTAC Model:

Both RNA-seq and proteomics data were downloaded from the Clinical Proteomics Tumor Analysis Consortium (CPTAC). T-tests were taken across reported histology groups (21 pairwise comparisons). To avoid unwanted dependencies in our data, repeat measurements from the same patient were removed from the study, keeping only the first collected sample. The first rather than last was selected in the hope that we could minimize confounding differences between cancer types with differences in treatment effects.

Similar tests were obtained with msTrawler using the Base Model for each protein:

$${y}_{ijk} = {β}_{0} + {α}_{i} + {b}_{j} + {ϵ}_{ijk} .$$

Once the bridge has been incorporated, as described above, β₀ represents the expected difference between the bridge and whichever cancer type is currently being used as the reference condition. i=1, . . . , 7 indexes the 7 cancer types and αᵢ=0 when the i'th cancer has been set to the reference. As before, bⱼ˜N(0, τ²) is a random effect that accounts for sample-to-sample variability which is independent from the within sample error, ∈ᵢⱼₖ˜N(0, σ²).

Results were matched up by gene name to the results from the CPTAC t-tests. In cases where msTrawler quantified multiple proteins from a single gene, the isoform with the smallest p-value across all comparisons was selected. Data were further reduced to only include genes quantified in all three methods (RNA-seq t-tests, CPTAC proteomics t-tests, and msTrawler).

### Example 16: Sample Preparation, Experimental Techniques, Pre-Processing

The below are summaries of materials, techniques, used in some of the examples herein:

Materials: Tandem mass tag (TMT) 16-plex isobaric reagents were obtained from Thermo Fisher Scientific (Rockford, IL). Modified trypsin was obtained from Promega Corporation (Madison, WI) and Lys-C from WAKO Chemicals (Richmond, VA). SepPak solid phase extraction cartridges were from Waters Corporation (Milford, MA). Complete protease inhibitor tablets were obtained from Roche. PCR Strip Caps (Axygen, 321-11-071) 0.2 mL Clear, For Real Time PCR, flat top. Platemax UltraClear Sealing Film (Axygen, UC-500). PCR Tubes—(Axygen, 321-02-501) 0.2 mL Maxymum Recovery, Thin Wall, Clear. Eppendorf 2.0 mL Protein LoBind tubes, Catalog #022431102. Eppendorf 1.5 mL Protein LoBind tubes, Catalog #02243108. Alpaqua 96-well neodymium magnet, Cat #A000400. Thermo Fisher Scientific Pierce Bicinchoninic Acid (BCA) Assay, 23228. GE Healthcare SpeedBead Magnetic Carboxylate Beads Hydrophilic and Hydrophobic forms (65152105050250, 45152105050250). Pierce FlexMix Calibration Solution, #A39239. Axygen 2 mL 96-well deep well plate, #P-2ML-SQ-C. Filtered, Conductive Hamilton Vantage™ Tips—1000 μL, 300 μL, 50 μL (#235940, #235938, #235979). Unless otherwise stated, all other chemicals were purchased from Sigma-Aldrich (St. Louis, MO).

### Experimental Techniques

Yeast Media: All media contained 1.71 g/L yeast nitrogen base (YNB, Sunrise Science) plus the indicated carbon and nitrogen sources. Final concentrations were 50 g/L ammonium sulfate, 23 g/L urea, 12.85 g/L monosodium glutamate, 3% glycerol and 2% ethanol, 20 g/L galactose, and 20 g/L glucose.

Growth Conditions: Strain CGY4.50 (BY-derived MATa prototroph) was struck from the freezer onto YPD plates (10 g/L yeast extract [Fisher], 20 g/L Bacto peptone [Fisher]20 g/L agar [Fisher]) and grown for 2 days at 30° C. For media containing glucose and galactose carbon sources, cells were inoculated into 3 mL of their respective media and grown overnight to saturation. Cells were diluted to an OD600 of 0.006 (˜1.2×105 cells/mL) in 10 mL and grown to an OD600 of ˜0.1 (˜2×106 cells/mL). They were then diluted into 250 mL to an initial OD of 0.002 for glucose+ammonium sulfate, glucose+urea, and glucose+monosodium glutamate, 0.01 for galactose+ammonium sulfate and galactose+monosodium glutamate, and 0.005 for galactose+urea and grown to a final OD of ˜0.3. For glycerol/ethanol+ammonium sulfate, cells were inoculated from a plate into 10 mL media, grown to saturation, diluted 1:1000 into 250 mL, and grown to a final OD of 0.5.

To harvest cells, cultures were poured over a 0.2 μM vacuum filter, washed with 50 mL water, collected with 5 mL water, spun down at 1,500×G for 5 minutes, resuspended in 1 mL water, and spun down again. The supernatant was removed, and the pellets were placed at −20° C.

Yeast Lysis: Yeast cells grown per different condition media were lysed using the GenoGrinder device (SpexSamplePrep), by adding 0.5 mL 8 M urea, 50 mM HEPES pH 8.5, vortex mixed, adding 0.5 mL 0.5 mm Zirconia/Silica beads and proceeding with lysis cycles. The lysis process consisted of cycling 5 times between 1 minute of shaking at 1500 rpm, followed by 30 seconds of no shaking. Blocks were pre-cooled to −20° C. prior to start.

Mouse plasma: Plasma was collected from C57/B6 mice purchased from The Jackson Laboratory. Mice were housed on a 12-hr light/dark cycle (lights on at 6:00, lights off at 18:00) in a temperature- and humidity-controlled environment (22±1° C., 60-70% humidity). Calico Life Sciences LLC is committed to the internationally accepted standard of the 3Rs (Reduction, Refinement, Replacement) and adhering to the highest standards of animal welfare in the company's research and development programs. Animal studies were approved by Calico's Institutional Animal Care and Use Committee or Ethics Committee. Animal studies were conducted in an AAALAC-accredited program where veterinary care and oversight was provided to ensure appropriate animal care. Mice were anesthetized by inhalation anesthesia using isoflurane (1.5-2.5%), 0.8-1.0 L/min oxygen flow rate). The blood samples were collected in EDTA tubes via cardiac puncture, the plasma was removed and stored at −80° C. for further processing.

### Sample Preparation

Lysed samples (8 M urea, 50 mM HEPES pH 8.5) were quantified for protein content using a Pierce BCA assay (Pierce, Thermo). For each of the seven yeast samples, 1 mg of yeast lysate (a total of 7 samples) was manually processed through digestion in addition to a single 17.5 mg aliquot of mouse (a grand total of 8 samples). In brief, samples were reduced using DTT (5 mM, 30 mins, RT) at room temperature, alkylated (15 mM IAA, RT in the dark) and quenched with an additional 5 mM DTT. Samples were then diluted into a final concentration of 4 M urea, 50 mM HEPES, pH 8.5, and digested overnight at room temperature while shaking at 600 rpm with LysC at a ratio of 1:50 protease:protein. Samples were then further diluted to 1 M urea 50 mM HEPES pH 8.5, and digested with trypsin at a ratio of 1:30 protease:protein, at 600 rpm, 37 C, 6 hours. Digestion was quenched by adding 10% trifluoroacetic acid (TFA) to achieve a final concentration of 0.5% TFA and centrifuged (17 k×g, 30 mins, 4° C.) to remove cellular debris. Digested peptides were purified using separate SepPak columns (Waters) at a ratio of 1:100 protein:resin. SepPak columns were first equilibrated with 1×1 mL 100% methanol, followed by equilibration with 1×1 mL 80% acetonitrile (ACN), followed by 1×1 mL wash of 0.5% acetic acid and 3×1 mL washes of 0.1% TFA. Acidified digest was then bound to column resin. This was followed by another 3×1 mL wash of 0.1% TFA and 1×1 mL wash of 0.5% acetic acid. Purified peptides were subsequently eluted with 1×0.75 mL 40% can, 0.5% acetic acid, and 1×0.75 mL 80% ACN, 0.5% acetic acid. Elutions were combined and dried down using a speedvac. Desalted peptides were resuspended in 200 mM HEPES pH 8.5 and quantified using BCA assay (Pierce, Thermo). The subsequent sample preparation steps were performed on the AutoMP3 platform28. For the entire 96-well plate, per well (with the exception of wells allocated for pooled bridge), 80 μg mouse plasma peptides were aliquoted and various media grown yeast spike-ins were performed to generate necessary dilution ratios using custom worklist directions for the Vantage liquid handler (Hamilton). Sample volumes were normalized to 80 μL with an additional 200 mM HEPES pH 8.5. At this point batch-bridges (population-level, and per-batch-level) were generated by taking a small portion out of each sample, pooled together, mixed, and aliquoted out into appropriate bridge well positions to complete 15-plex batches. Non-bridge sample volumes were again normalized to 80 μL with additional 200 mM HEPES pH 8.5. All samples were then TMT labeled with automated addition of TMTpro29 at 8:1 TMT:peptide ratio. Samples were briefly mixed and incubated for 1 hour at room temperature. Samples were then quenched by addition of 11 μL 5% hydroxylamine, mixed, incubated at room temperature for 15 minutes, re-combined by batch, partially dried down to remove acetonitrile, and acidified with trifluoroacetic acid to a final concentration of 0.5%. Each batch was then desalted using C18 StageTips30, with peptides eluted using 40% acetonitrile/5% formic acid, and then 80% acetonitrile/5% formic acid and dried down under a vacuum at room temperature (Labconco CentriVap Benchtop Vacuum Concentrator, Kansas City, Mo). Samples were then resuspended in 5% ACN/5% Formic acid at a concentration of 0.5 ug/μL, and approximately 1 μg per sample was injected for analysis on an Orbitrap Eclipse as described below.

For the Ratio Expansion Experiment, samples were prepared as above with the following modifications. PC9 human cell line was prepared and used as the background species. A batch of Saccharomyces cerevisiae, apart from The Interbatch Experiment, was grown in YPD+G418 media and the subsequent lysate was diluted to generate target ratios, as before. Samples were TMT labeled with 18-plex TMTpro instead of 16-plex to accommodate plex design.

### LC-MS Analysis

Peptides were analyzed on an Orbitrap Eclipse mass spectrometer coupled to an Ultimate 3000 (Thermo Fisher Scientific). Peptides were separated on an IonOpticks Aurora microcapillary column (75 μm inner diameter, 25 cm long, C18 resin, 1.6 μm, 120 Å). The total LC-MS run length for each sample was 185 min including a 165 min gradient from 6 to 35% ACN in 0.1% formic acid. The flow rate was 300 nL/min, and the column was heated at 60° C.

We used Real Time Search (RTS)31 and Field Asymmetric Ion Mobility Spectroscopy device (FAIMS Pro) connected to the Orbitrap Eclipse mass spectrometer. We set up a method with four experiments, where each experiment utilized a different FAIMS Pro compensation voltage: −40, −50, −60, and −70 Volts. Each of the four experiments had a 1.25 second cycle time. A high resolution MS1 scan in the Orbitrap (m/z range 400-1,600, 120 k resolution, AGC 4×105, “Auto” max injection time, ion funnel RF of 30%) was collected from which the top 10 precursors were selected for MS2 followed by SPS MS3 analysis. For MS2 spectra, ions were isolated with the quadrupole mass filter using a 0.7 m/z isolation window. The MS2 product ion population was analyzed in the quadrupole ion trap (CID, AGC 1×104, normalized collision energy 35%, “Auto” max injection time) and the MS3 scan was analyzed in the Orbitrap (HCD, 50 k resolution, AGC 1×105, max injection time 200 ms, normalized collision energy 45%). Up to ten fragment ions from each MS2 spectrum were selected for MS3 analysis using SPS. A combined yeast and mouse database was used for RTS and a maximum of two missed cleavages and one variable modification was allowed. The maximum search time was set to 35 ms and a Xcorr of 1, dCN 0.1 and precursor of 10 ppm for charge state 2, 3 and 4 was used.

Settings were the same for the Ratio Expansion Experiment except that the samples were analyzed with two SPS precursor ranges, 400-1600 and 455-1600 mz, using the same parameters as above. The reason for these two ranges was to evaluate the effect of the inclusion of the y1 ion.

### Peptide Identification and Quantification

Mass spectrometry data were processed using an previously described software pipeline32. Raw files were converted to mzXML files and searched against either a composite mouse, yeast* Uniprot/SGD* databases (downloaded on Jun. 10, 2020), in forward and reverse orientations using the Comet algorithm. Database searching matched MS/MS spectra with fully tryptic peptides from this composite dataset with a precursor ion tolerance of 20 p.p.m., fragment_bin_tol set to 1.0005 and fragment_bin_offset=0.4 Da. TMTpro tags on peptide N-termini and lysines (+304.20 Da) were set as static modifications. Oxidation of methionine (+15.99 Da) was set as a variable modification. Linear discriminant analysis was used to filter peptide spectral matches to a 1% FDR (false discovery rate) as described previously33. Non-unique peptides that matched to multiple proteins were assigned to proteins that contained the largest number of matched redundant peptide sequences using the principle of Occam's razor33. However, in the interest of completely separating mouse and yeast peptides we subsequently removed all non-unique peptides from the analysis. Quantification of TMT reporter ion intensities was performed by extracting the most intense ion within a 0.003 m/z window at the predicted m/z value for each reporter ion.

Mass spectra for the Ratio Expansion Experiment were interpreted with Proteome Discover v3.0.1.27 (Thermo Fischer Scientific). In brief, the parent mass error tolerance was set to 20 ppm and the fragment mass error tolerance to 0.6 Da. Strict trypsin specificity was required allowing for up to two missed cleavages. Carbamidomethylation of cysteine (+57.021 Da), TMT-labeled N terminus and lysine (+229.163) were set as static modifications. Methionine oxidation (+15.995); and N-terminal acetylation (+42.011) were set as variable modifications. The minimum required peptide length was set to six amino acids. Spectra were queried against a “target-decoy” protein sequence database consisting of human and yeast proteins, contaminants, and reversed decoys of the above using the SEQUEST algorithm34. The Percolator algorithm35 was used to estimate and remove false-positive identifications to achieve a strict false discovery rate of 1% at both peptide and protein levels. The coisolation threshold was set to 0, average reporter signal to noise was set to 10, SPS mass matches was set to (65%), and everything was evaluated with and without isotopic purity correction of reporter quant values. The PSMS tables were exported and further analysis was performed in R.

### Data Pre-Processing and Modeling

Numerous steps were implemented to filter the data prior to starting the analysis described in Example 3. First, peptides that were identified as reverse hits or contaminants were removed. To decrease the chance of a peptide being assigned to the wrong species, any peptide that matched to more than one protein was removed. Scans with almost no signal at all were also removed by looking for cases with fewer than 3 non-zero intensities or with a summed signal-to-noise ratio less than 20. Additionally, a Pearson correlation was calculated between each scan and the corresponding yeast dilutions. All peptides with an identification assigned to a mouse protein but with a correlation greater than 0.25 were removed.

For the tumor analysis, but not the benchmarking experiment, outlier detection from the msTrawler package was also used to remove scans that were far different from a consensus pattern within each protein. The procedure takes one batch at a time and computes the centered log 2 ratio for each scan. For each protein, standardized residuals were calculated from a linear model estimating an average normalized intensity for each sample. Any scan with a standardized residual greater than 4, in any of the samples, was removed. Although the feature was not used in this study, by default msTrawler will remove outliers from protein groupings by replacing the protein name with a peptide name, allowing us to associate the outliers with parameters in the experiment that might be driven by post-translational modifications. Outlier detection was not used in the benchmarking experiment since some of the methods we were comparing against utilize all scans, in particular the SUM method), and we wanted to see performance metrics on equivalent sets of data.

Global column adjustments were also performed using the functions from the MsTrawler package, as described in Appendix 5. For our benchmarking experiment, the 50th percentile was calculated only after excluding yeast proteins from the analysis (since only mouse proteins should be equal across each sample). Additionally, to make this dataset compatible with MSstats-TMT, repeat peptides were removed (keeping only the scan with the highest summed intensities). These intermediate data files, and another utilizing a SSN filter of 200, are available in the supplementary data and code.

Mixed models are fit using the lme4 package for the R programming language36 and Kenward-Roger corrections are implemented using the pbkrtest package37. When multi-level modeling is not possible, primarily when we have only a single observation per sample, the random effects are removed, and the model is reduced to weighted least squares. Additionally, proteins with large numbers of repeat observations can cause an unnecessary computational burden, so we placed a ceiling of 25 on the number of peptides allowed per protein (in each sample). To meet this requirement while preserving peptide diversity, we took the 25 scans with the highest SSN without allowing any repeat measurements until all observed peptide species had been selected. Code for this algorithm, and the others described in this section, can be found in the MsTrawler package.

Particular embodiments of the subject matter have been described. Other embodiments are within the scope of the following claims. For example, the actions recited in the claims can be performed in a different order and still achieve desirable results. As one example, the processes depicted in the accompanying figures do not necessarily require the particular order shown, or sequential order, to achieve desirable results. In certain implementations, multitasking and parallel processing may be advantageous. Other steps or stages may be provided, or steps or stages may be eliminated, from the described processes. Accordingly, other implementations are within the scope of the following claims.

It will be appreciated to those skilled in the art that the preceding examples and embodiments are exemplary and not limiting to the scope of the present disclosure. It is intended that all permutations, enhancements, equivalents, combinations, and improvements thereto that are apparent to those skilled in the art upon a reading of the specification and a study of the drawings are included within the true spirit and scope of the present disclosure. It shall also be noted that elements of any claims may be arranged differently including having multiple dependencies, configurations, and combinations.

Having thus described several aspects of at least one embodiment of this invention, it is to be appreciated that various alterations, modifications, and improvements will readily occur to those skilled in the art. Such alterations, modifications, and improvements are intended to be part of this disclosure, and are intended to be within the spirit and scope of the invention. Accordingly, the foregoing description and drawings are by way of example only.

What is claimed is:

1. A method of measuring amounts of one or more peptides in a plurality of batches, each batch comprising a plurality of samples, each sample comprising one or more labeled peptides, the method comprising:
a) performing, with a mass spectrometer, quantitative mass spectroscopy on the plurality of batches, thereby obtaining multiplexed mass spectrometry proteomics data (“MSPD”);
b) obtaining, from the MSPD, one or both of i) reporter ion intensities or a derivative thereof (“intensities”) and ii) reporter ion signal-to-noise ratios or a derivative thereof (“SNRs”) for each peptide in a given sample, wherein the intensities SNRs correspond to one or more scans performed on the given sample;
c) receiving, for each scan of the one or more scans in each sample, corresponding covariate values for one or more covariates;
d) for each respective parameter of one or more parameters of a statistical model, performing a computation to estimate the respective parameter, each of the one or more parameters representing an association between i) intensities of at least one respective peptide of the one or more peptides and ii) the covariates, the computation comprising:
i) appending a design matrix from the statistical model to incorporate intensities from a bridge sample to allow estimating one or more scan specific nuisance variables, the bridge sample representing a pooled sample from each of the one or more batches;
ii) weighting the intensities based on the corresponding SNR;
iii) fitting the statistical model to the weighted intensities; and
iv) estimating a value of the parameter and one or more p-values of one or more hypothesis tests for the parameter; and
e) reporting, based on the estimated values of the one or more parameters of the statistical model, the amounts of the one or more peptides in each of the samples.

2. The method of claim 1, wherein the one or more peptides correspond to a protein.

3. The method of claim 1 or 2, wherein the computation further comprises identifying one or more of the parameters to be estimable based on the intensities and the statistical model, wherein outputting the estimate of the parameter and the one or more p-values corresponds to an estimable parameter.

4. The method of any one of claims 1 to 3, further comprising identifying any intensities for a respective scan in a given sample that has an intensity less than a threshold, wherein weighting said intensities for each of said identified intensities comprises a down weighted value instead of the corresponding SNR or derivative thereof.

5. The method of claim 4, wherein the threshold is a percentage of a total summed signal of intensities in a given batch.

6. The method of claim 5, wherein the percentage is at most about 0.5%, 1%, 1.5%, 2%, or 3%.

7. The method of any one of claims 1 to 6, further comprising removing any outliers identified with the intensities and/or SNR.

8. The method of any one of claims 1 to 7, wherein the covariate values correspond to the number of parameters of the statistical model.

9. The method of any one of claims 1 to 8, wherein each covariate comprises a covariate factor, a continuous covariate, and/or a time trend within one or more levels of a factor.

10. The method of claim 9, wherein the time trends comprise a linear time trend, a cubic time trend, a quadratic time trend, a circadian time trend, or any combination thereof.

11. The method of any one of claims 8 to 10, wherein the covariate corresponds to an environmental condition and/or a characteristic of a subject from where a peptide was obtained.

12. The method of claim 11, wherein the environmental condition comprises a media type for a sample, a dilution factor for a peptide or the protein, a temperature of the sample, or any combination thereof.

13. The method of claim 11 or 12, wherein the characteristic of a subject comprises an age of the subject, an ethnicity of the subject, a sex of the subject, a height of the subject, a weight of the subject, a physical attributed of the subject, a medical diagnosis of the subject, the subject being administered a treatment, the subject intaking a medication, a location for the protein, a type of medical condition, a cell type, or any combination thereof.

14. The method of claim 13, wherein the location of the targeted protein comprises a tissue of the subject.

15. The method of claim 14, wherein the tissue comprises a brain, a lung, a heart, a skin, a liver, a stomach, or any combination thereof.

16. The method of any one of claims 1 to 15, wherein the covariate comprises a covariate factor, wherein the covariate values for the covariate factor identifies a number of levels pertaining to the factor.

17. The method of any one of claims 1 to 16, wherein the covariate comprises a continuous factor, wherein the covariate values for the continuous covariate identifies a numerical value.

18. The method of any one of claims 1 to 17, wherein the statistical model further comprises a sample identification parameter that distinguishes a plurality of samples based on the same source, so as to account for variance between the plurality of samples.

19. The method of claim 18, wherein the sample identification parameter is configured to fit the design matrix and/or the appended design matrix to a longitudinal model.

20. The method of any one of claims 1 to 19, wherein the statistical model is a multi-level model to account for correlations between intensities of a same sample.

21. The method of claim 20, further comprising adjusting a p-value of the one or more p-values to account for small sample sizes.

22. The method of claim 21, wherein adjusting the p-value comprises using Kenward-Roger corrections.

23. The method of any one of claims 1 to 22, wherein each scan specific nuisance variable corresponds to a scan to scan variation between two or more batches.

24. A non-transitory computer readable medium for processing multiplexed mass spectrometry proteomics data (“MSPD”) from a plurality of batches, each batch comprising a plurality of samples that each comprise one or more peptides, the non-transitory computer readable medium comprising instructions that, when executed by a processor, cause the processor to perform operations including:
a) receiving, from the MSPD, one or both of i) reporter ion intensities or a derivative thereof (“intensities”) and ii) reporter ion signal-to-noise ratios or a derivative thereof (“SNRs”) for each peptide in a given sample, wherein the intensities SNRs correspond to one or more scans performed on the given sample;
b) receiving, for each scan of the one or more scans in each sample, corresponding covariate values for one or more covariates;
c) for each respective parameter of one or more parameters of a statistical model, performing a computation to estimate the respective parameter, each of the one or more parameters representing an association between i) intensities of at least one respective peptide of the one or more peptides and ii) the covariates, the computation comprising:
i) appending a design matrix from the statistical model to incorporate intensities from a bridge sample to allow estimating one or more scan specific nuisance variables, the bridge sample representing a pooled sample from each of the one or more batches;
ii) weighting the intensities based on the corresponding SNR;
iii) fitting the statistical model to the weighted intensities; and
iv) outputting an estimate of the parameter and one or more p-values of one or more hypothesis tests for the parameter.

25. The non-transitory computer readable medium of claim 24, wherein the one or more peptides correspond to a protein.

26. The non-transitory computer readable medium of claim 24 or 25, wherein the computation further comprises identifying one or more of the parameters to be estimable based on the intensities and the statistical model, wherein outputting the estimate of the parameter and the one or more p-values corresponds to an estimable parameter.

27. The non-transitory computer readable medium of any one of claims 24 to 26, wherein the operations further includes identifying any intensities for a respective scan in a given sample that has an intensity less than a threshold, wherein weighting said intensities for each of said identified intensities comprises a down weighted value instead of the corresponding SNR or derivative thereof.

28. The non-transitory computer readable medium of claim 27, wherein the threshold is a percentage of a total summed signal of intensities in a given batch.

29. The non-transitory computer readable medium of claim 28, wherein the percentage is at most about 0.5%, 1%, 1.5%, 2%, or 3%.

30. The non-transitory computer readable medium of any one of claims 24 to 29, wherein the operations further includes removing any outliers identified with the intensities and/or SNR.

31. The non-transitory computer readable medium of any one of claims 24 to 30, wherein the covariate values correspond to the number of parameters of the statistical model.

32. The non-transitory computer readable medium of any one of claims 24 to 31, wherein each covariate comprises a covariate factor, a continuous covariate, and/or a time trend within one or more levels of a factor.

33. The non-transitory computer readable medium of claim 32, wherein the time trends comprise a linear time trend, a cubic time trend, a quadratic time trend, a circadian time trend, or any combination thereof.

34. The non-transitory computer readable medium of any one of claims 31 to 33, wherein the covariate corresponds to an environmental condition and/or a characteristic of a subject from where a peptide was obtained.

35. The non-transitory computer readable medium of claim 34, wherein the environmental condition comprises a media type for a sample, a dilution factor for a peptide or the protein, a temperature of the sample, or any combination thereof.

36. The non-transitory computer readable medium of claim 34 or 35, wherein the characteristic of a subject comprises an age of the subject, an ethnicity of the subject, a sex of the subject, a height of the subject, a weight of the subject, a physical attributed of the subject, a medical diagnosis of the subject, the subject being administered a treatment, the subject intaking a medication, a location for the protein, a type of medical condition, a cell type, or any combination thereof.

37. The non-transitory computer readable medium of claim 36, wherein the location of the targeted protein comprises a tissue of the subject.

38. The non-transitory computer readable medium of claim 37, wherein the tissue comprises a brain, a lung, a heart, a skin, a liver, a stomach, or any combination thereof.

39. The non-transitory computer readable medium of any one of claims 24 to 38, wherein the covariate comprises a covariate factor, wherein the covariate values for the covariate factor identifies a number of levels pertaining to the factor.

40. The non-transitory computer readable medium of any one of claims 24 to 39, wherein the covariate comprises a continuous factor, wherein the covariate values for the continuous covariate identifies a numerical value.

41. The non-transitory computer readable medium of any one of claims 24 to 40, wherein the statistical model further comprises a sample identification parameter that distinguishes a plurality of samples based on the same source, so as to account for variance between the plurality of samples.

42. The non-transitory computer readable medium of claim 41, wherein the sample identification parameter is configured to fit the design matrix and/or the appended design matrix to a longitudinal model.

43. The non-transitory computer readable medium of any one of claims 24 to 42, wherein the statistical model is a multi-level model to account for correlations between intensities of a same sample.

44. The non-transitory computer readable medium of claim 43, wherein the operations further includes adjusting a p-value of the one or more p-values to account for small sample sizes.

45. The non-transitory computer readable medium of claim 44, wherein adjusting the p-value comprises using Kenward-Roger corrections.

46. The non-transitory computer readable medium of any one of claims 24 to 45, wherein each scan specific nuisance variable corresponds to a scan to scan variation between two or more batches.

47. A method for processing multiplexed mass spectrometry proteomics data (“MSPD”) from a one or more batches, each batch comprising a plurality of samples that each comprise one or more peptides, the method comprising:
a) receiving, from the MSPD, one or both of i) reporter ion intensities or a derivative thereof (“intensities”) and ii) reporter ion signal-to-noise ratios or a derivative thereof (“SNRs”) for each peptide in a given sample, wherein the intensities SNRs correspond to one or more scans performed on the given sample;
b) receiving, for each scan of the one or more scans in each sample, corresponding covariate values for one or more covariates;
c) for each respective parameter of one or more parameters of a statistical model, performing a computation to estimate the respective parameter, each of the one or more parameters representing an association between i) intensities of at least one respective peptide of the one or more peptides and ii) the covariates, the computation comprising:
i) weighting the intensities based on the corresponding SNR;
ii) fitting the statistical model to the weighted intensities; and
iii) outputting an estimate of the parameter and one or more p-values of one or more hypothesis tests for the parameter.

48. The method of claim 47, wherein the one or more peptides correspond to a protein.

49. The method of claim 47 or 48, wherein the computation further comprises identifying one or more of the parameters to be estimable based on the intensities and the statistical model, wherein outputting the estimate of the parameter and the one or more p-values corresponds to an estimable parameter.

50. The method of any one of claims 47 to 49, further comprising identifying any intensities for a respective scan in a given sample that has an intensity less than a threshold, wherein weighting said intensities for each of said identified intensities comprises a down weighted value instead of the corresponding SNR or derivative thereof.

51. The method of claim 50, wherein the threshold is a percentage of a total summed signal of intensities in a given batch.

52. The method of claim 51, wherein the percentage is at most about 0.5%, 1%, 1.5%, 2%, or 3%.

53. The method of any one of claims 47 to 52, further comprising removing any outliers identified with the intensities and/or SNR.

54. The method of any one of claims 47 to 53, wherein the covariate values correspond to the number of parameters of the statistical model.

55. The method of any one of claims 47 to 54, wherein each covariate comprises a covariate factor, a continuous covariate, and/or a time trend within one or more levels of a factor.

56. The method of claim 55, wherein the time trends comprise a linear time trend, a cubic time trend, a quadratic time trend, a circadian time trend, or any combination thereof.

57. The method of any one of claims 54 to 56, wherein the covariate corresponds to an environmental condition and/or a characteristic of a subject from where a peptide was obtained.

58. The method of claim 57, wherein the environmental condition comprises a media type for a sample, a dilution factor for a peptide or the protein, a temperature of the sample, or any combination thereof.

59. The method of claim 57 or 58, wherein the characteristic of a subject comprises an age of the subject, an ethnicity of the subject, a sex of the subject, a height of the subject, a weight of the subject, a physical attributed of the subject, a medical diagnosis of the subject, the subject being administered a treatment, the subject intaking a medication, a location for the protein, a type of medical condition, a cell type, or any combination thereof.

60. The method of claim 59, wherein the location of the targeted protein comprises a tissue of the subject.

61. The method of claim 60, wherein the tissue comprises a brain, a lung, a heart, a skin, a liver, a stomach, or any combination thereof.

62. The method of any one of claims 47 to 61, wherein the covariate comprises a covariate factor, wherein the covariate values for the covariate factor identifies a number of levels pertaining to the factor.

63. The method of any one of claims 47 to 62, wherein the covariate comprises a continuous factor, wherein the covariate values for the continuous covariate identifies a numerical value.

64. The method of any one of claims 47 to 63, wherein the statistical model further comprises a sample identification parameter that distinguishes a plurality of samples based on the same source, so as to account for variance between the plurality of samples.

65. The method of claim 64, wherein the sample identification parameter is configured to fit the design matrix to a longitudinal model.

66. The method of any one of claims 47 to 63, wherein the statistical model is a multi-level model to account for correlations between intensities of a same sample.

67. The method of claim 66, further comprising adjusting a p-value of the one or more p-values to account for small sample sizes.

68. The method of claim 67, wherein adjusting the p-value comprises using Kenward-Roger corrections.

69. A method for processing multiplexed mass spectrometry proteomics data (“MSPD”) from a plurality of batches, each batch comprising a plurality of samples that each comprise one or more peptides, the method comprising:
a) obtaining, from the MSPD, one or both of i) reporter ion intensities or a derivative thereof (“intensities”) and ii) reporter ion signal-to-noise ratios or a derivative thereof (“SNRs”) for each peptide in a given sample, wherein the intensities SNRs correspond to one or more scans performed on the given sample;
b) receiving, for each scan of the one or more scans in each sample, corresponding covariate values for one or more covariates;
c) for each respective parameter of one or more parameters of a statistical model, performing a computation to estimate the respective parameter, each of the one or more parameters representing an association between i) intensities of at least one respective peptide of the one or more peptides and ii) the covariates, the computation comprising:
i) appending a design matrix from the statistical model to incorporate intensities from a bridge sample to allow estimating one or more scan specific nuisance variables, the bridge sample representing a pooled sample from each of the one or more batches;
ii) weighting the intensities based on the corresponding SNR;
iii) fitting the statistical model to the weighted intensities; and
iv) outputting an estimate of the parameter and one or more p-values of one or more hypothesis tests for the parameter.

70. The method of claim 69, wherein the one or more peptides correspond to a protein.

71. The method of claim 69 or 70, wherein the computation further comprises identifying one or more of the parameters to be estimable based on the intensities and the statistical model, wherein outputting the estimate of the parameter and the one or more p-values corresponds to an estimable parameter.

72. The method of any one of claims 69 to 71, further comprising identifying any intensities for a respective scan in a given sample that has an intensity less than a threshold, wherein weighting said intensities for each of said identified intensities comprises a down weighted value instead of the corresponding SNR or derivative thereof.

73. The method of claim 72, wherein the threshold is a percentage of a total summed signal of intensities in a given batch.

74. The method of claim 73, wherein the percentage is at most about 0.5%, 1%, 1.5%, 2%, or 3%.

75. The method of any one of claims 69 to 74, further comprising removing any outliers identified with the intensities and/or SNR.

76. The method of any one of claims 69 to 75, wherein the covariate values correspond to the number of parameters of the statistical model.

77. The method of any one of claims 69 to 76, wherein each covariate comprises a covariate factor, a continuous covariate, and/or a time trend within one or more levels of a factor.

78. The method of claim 77, wherein the time trends comprise a linear time trend, a cubic time trend, a quadratic time trend, a circadian time trend, or any combination thereof.

79. The method of any one of claims 76 to 78, wherein the covariate corresponds to an environmental condition and/or a characteristic of a subject from where a peptide was obtained.

80. The method of claim 79, wherein the environmental condition comprises a media type for a sample, a dilution factor for a peptide or the protein, a temperature of the sample, or any combination thereof.

81. The method of claim 79 or 80, wherein the characteristic of a subject comprises an age of the subject, an ethnicity of the subject, a sex of the subject, a height of the subject, a weight of the subject, a physical attributed of the subject, a medical diagnosis of the subject, the subject being administered a treatment, the subject intaking a medication, a location for the protein, a type of medical condition, a cell type, or any combination thereof.

82. The method of claim 81, wherein the location of the targeted protein comprises a tissue of the subject.

83. The method of claim 82, wherein the tissue comprises a brain, a lung, a heart, a skin, a liver, a stomach, or any combination thereof.

84. The method of any one of claims 69 to 83, wherein the covariate comprises a covariate factor, wherein the covariate values for the covariate factor identifies a number of levels pertaining to the factor.

85. The method of any one of claims 69 to 84, wherein the covariate comprises a continuous factor, wherein the covariate values for the continuous covariate identifies a numerical value.

86. The method of any one of claims 69 to 85, wherein the statistical model further comprises a sample identification parameter that distinguishes a plurality of samples based on the same source, so as to account for variance between the plurality of samples.

87. The method of claim 86, wherein the sample identification parameter is configured to fit the design matrix and/or the appended design matrix to a longitudinal model.

88. The method of any one of claims 69 to 87, wherein the statistical model is a multi-level model to account for correlations between intensities of a same sample.

89. The method of claim 88, further comprising adjusting a p-value of the one or more p-values to account for small sample sizes.

90. The method of claim 89, wherein adjusting the p-value comprises using Kenward-Roger corrections.

91. The method of any one of claims 69 to 90, wherein each scan specific nuisance variable corresponds to a scan to scan variation between two or more batches.
